(19) United States
(12) Patent Application Publication
(10) Pub. No.: US 2026/0242397 A1
(43) Pub. Date: Aug. 20, 2026

(54) THERMALLY ACTIVATED DELAYED FLUORESCENT SOLVENT-FREE ORGANIC LIQUID

(71) Applicant: COUNCIL OF SCIENTIFIC & INDUSTRIAL RESEARCH, New Delhi (IN)

(72) Inventors: Goudappagouda _, Pune (IN); Aakash Deepakarao NIDHANKAR, Pune (IN); Santhosh Babu SUKUMARAN, Pune (IN)

(21) Appl. No.: 18/724,886

(22) PCT Filed: Jan. 13, 2023
(86) PCT No.: PCT/IN2023/050032

§ 371 Date: Jun. 27, 2024

(30) Foreign Application Priority Data
Jan. 13, 2022 (IN) 202211002220

(51) Int. Cl. C07F 5/04; C07D 221/06; C07D 401/12; C07D 417/12

(52) U.S. Cl. CPC C07F 5/04; C07D 221/06; C07D 401/12; C07D 417/12

(57) ABSTRACT

Thermally activated delayed fluorescent solvent-free organic liquid compounds having formula (I) exhibits thermally activated delayed fluorescence properties for tunable emission and 3D-printing applications. Methods for preparing the compounds of formula (I) include reacting a brominated compound with a reactant in a reaction mixture including a base, a solvent, and optionally a catalyst and an additional ingredient at 100° C. to 150° C. for 9 hours to 28 hours.

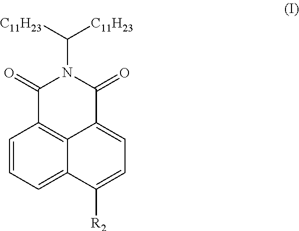

### CROSS-REFERENCES TO RELATED APPLICATIONS

This application is a national-stage application under 35 U.S.C. § 371 of International Application No. PCT/IN2023/050032, filed Jan. 13, 2023, which International Application claims benefit of priority to Indian Patent Application number 202211002220, filed Jan. 13, 2022.

### TECHNICAL FIELD

The present disclosure relates to thermally activated delayed fluorescent solvent-free organic liquid hybrids of Formula (I) and Formula (II) for tunable emission and 3D-printing applications.

### BACKGROUND

The conventional fluorescent emitters are emissive in the solution state and on solvent volatilization the emission property however gets severely affected due to quenching of emission by aggregation and photo-bleaching. Though conventional fluorescent emitters are preferred due to the comparatively high freedom and simplicity in molecular design, the inherently reduced internal quantum efficiency (25%) of conventional fluorescent organic light-emitting diodes (OLEDs) and the concerns related to the devise stability of phosphorescent LEDs has urged for thermally activated delayed fluorescence (TADF) sensitized devices. As a replacement, non-volatile luminescent materials such as crystals, ionic liquid crystals, and ionic liquids have been experimented. However, these materials are associated with the issues related to synthesis and purification in the bulk scale. Further, the emitters in conventional LED gives electric excitation, wherein, one pair of electron is formed, collides and takes only 25% of the excitation formed, because only singlet is involved, 75% which is triplet is unexplored. The fluorescence occurs in nano seconds.

To overcome, phosphorescence was explored, where both single and triplet can be used, singlet is converted to triplet as crossing occurs, so 100% internal quantum efficiency is obtained, however, the device efficiency decreases because of delay of few milli seconds or seconds.

Amorphous room temperature phosphorescence (RTP) liquid with enhanced phosphorescence features through molecular recognition by doping with carbonyl guests were reported recently in the article titled “Paintable Room-Temperature Phosphorescent Liquid Formulations of Alkylated Bromonaphthalimide” by Goudappagouda, A. Manthanath et. al published in Angew. Chem. Int. Ed. 2019, 58, 2284. Hence, the technologically advanced lighting and display applications demand for newer emitters with improved processability and luminescence features. Thermally activated delayed fluorescence (TADF) was therefore developed wherein, the singlet is converted to triplet, when a gap in energy is small and reverse intersystem crossing occurs, triplet goes back to singlet and both are used and the lifetime is in microseconds.

Thus, to decrease the gap further TADF is doped with a host which transfers energy from host to another molecule, which will emit light as tuned in white, blue or green regions, depending on wave length, the lifetime is improved with the reverse inter system crossing occurring at room temperature. Drawback of crystalline TADF material as reported in the art is having an inherent difficulty of preparing homogeneous thin films for OLEDs, hence, making it more inefficient, and not preferable at all.

The solvent-free organic liquids (SOLs) are introduced as a new soft material to the balance between efficiency and processability that is currently faced by conventional emitters. To meet the challenges in the art, the present inventors have introduced a TADF liquid and efficient energy transfer (ET)-assisted tunable emission by doping with TADF and fluorescent emitters to improve the device performance by suppressing the efficiency roll-off and reducing the density of triplet excitons of the host.

The primary objective of the present disclosure is to provide a solvent-free organic liquid (SOL) having attractive TADF features in bulk along with high processability and to the process of synthesis thereof.

### SUMMARY

In accordance with the above, the present disclosure provides thermally activated delayed fluorescent solvent free organic liquid having the compound of Formula (I). The present disclosure also discloses a method of preparation of solvent free organic liquid compound of formula (I). In an aspect, the present disclosure relates to solvent free organic liquid which exhibits thermally activated delayed fluorescence property (TADF) comprising the compound of Formula (I),

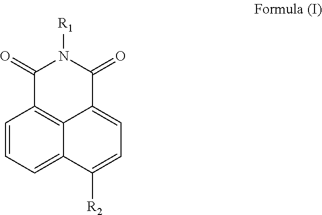

### Wherein;

R₁ is

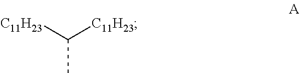

R₂ are independently selected from the group consisting of

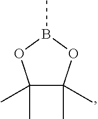

—O—R₃, —NR₄R₅, -Ph-NR₈R₉;

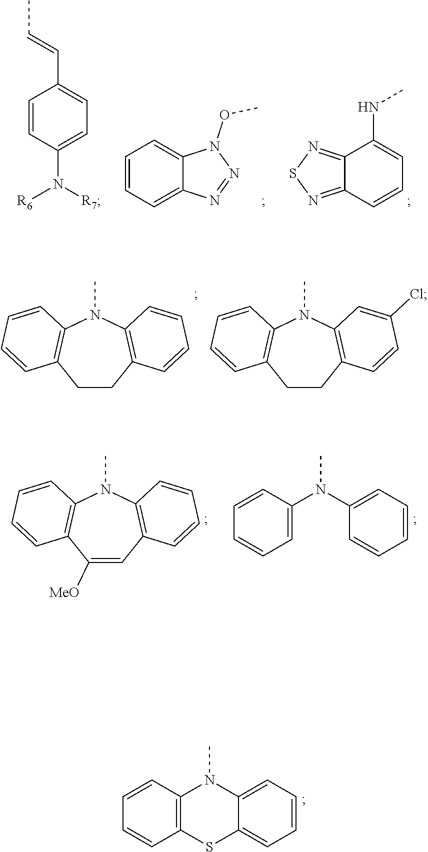

and

R₃, R₄, R₅, R₆, R₇, R₈, and R₉ are independently selected from the group consisting of (un)substituted linear or branched alkyl, (un)substituted linear or branched alkylene or (un)substituted linear or branched alkynyl, (un)substituted aryl or heteroaryl.

In another aspect, the present disclosure provides the process for preparation of the compounds of Formula (I) and Formula (II).

In another aspect, compounds of Formula (I) and Formula (II) may be in solid or liquid form.

In another aspect, the present disclosure provides the formulation comprising the compounds of Formula (I) and Formula (II) together with suitable formulating excipients.

In another aspect, the present disclosure relates to thermally activated delayed fluorescence (TADF) emitters compounds comprising phthalimide and naphthalimide materials for OLED application.

### BRIEF DESCRIPTION OF THE FIGURES

FIG. 1A depicts the DSC thermograms of compounds 1-5 at a scanning rate of 10° C./min, showing the T_{goffset} values, FIG. 1B depicts TGA of compounds 1-5 at a scanning rate of 10° C./min, showing the T_{goffset} values.

FIG. 2 depicts the Variation of a) loss modulus (Gⁿ) (circles), and storage modulus (G′) (squares), b) complex viscosity (η*) versus angular frequency on double logarithmic scale, of compound (1). c) Photographs of the free-flowing liquid feature of compound (1).

FIG. 3 depicts the Fluorescence lifetime decay profiles of 1-5 in a) in MTHF solution (C=1×10⁻⁵ M) and b) in neat state at 298 K (λₑₓ=374 nm, λₘₒₙ=420 nm for 1,420 nm for 2, 524 nm for 3, 564 nm for 4, 620 nm for 5).

FIG. 4 depicts the TADF and RTP spectra of compound (1) in neat state at 298 K showing ΔE_{ST} of 0.0798 eV (λₑₓ=355 nm).

FIGS. 5A, 5B, 5C, 5D, 5E depict the HOMO-LUMO and energy level diagram of 1-5 showing ΔE_{ST} for compounds (a) to (e), respectively, from DFT calculations.

FIG. 6 depicts a Comparison of the emission spectra in of 1-5 in neat state at 298 K and 77 K (λₑₓ=355 nm for 1, λₑₓ=360 nm for 2, λₑₓ=424 nm for 3, λₑₓ=434 nm for 4, λₑₓ=480 nm for 5).

FIG. 7 depicts the Spectra overlap integral of emission and absorption of a)₁ and 3, b)₁ and 4, c)₁ and 4, d)₂ and 3, e)₂ and 4, f)₂ and 5, respectively, in MTHF solution.

FIG. 8 is a Photograph of the SOL (1-5)-PLA hybrid sheets under visible (top) and UV (365 nm) (bottom) lights, and b) a cube with different emission colors (1-5), including white (2+5) (1:0.002) made out of hybrid sheets under UV (365 nm) light. Photographs of the 3D printed objects using PLA-TADF SOL (1 wt %) hybrids under UV light (365 nm). c) CSIR and NCL logo using 1-PLA and d) colorful flower vase (2-5 and 2+5) and mushroom lamp (2, 5).

FIG. 9 Scheme 1: Synthesis of compound (1-3).

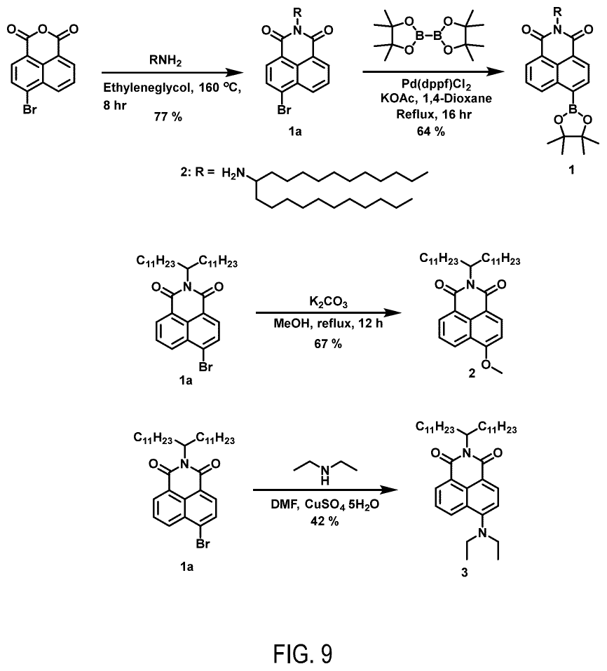

FIG. 10 Scheme 2: Synthesis of compound (4).

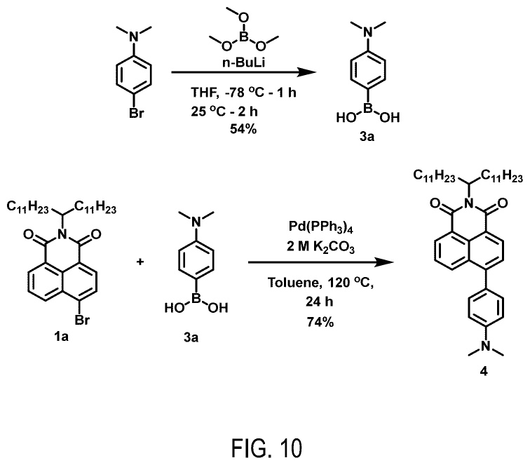

FIG. 11 Scheme 3: Synthesis of compound (5)

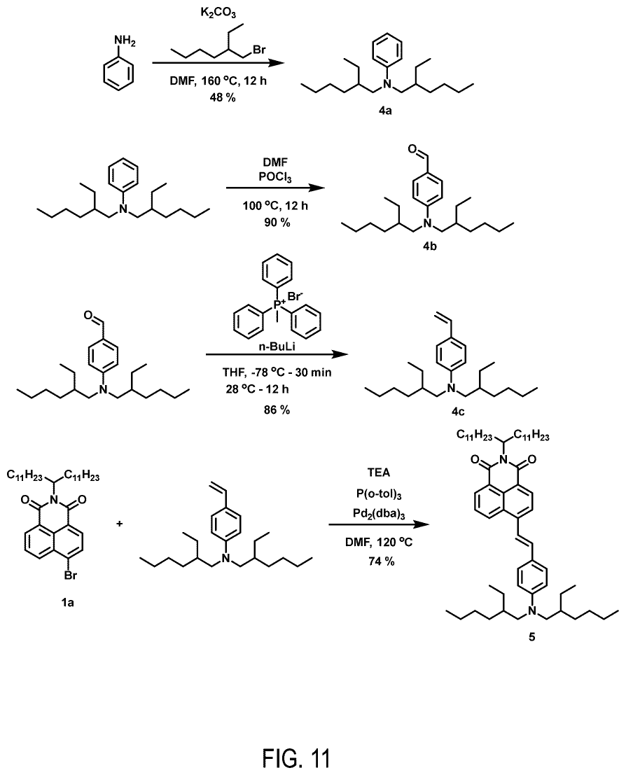

### DETAILED DESCRIPTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the scope of the present disclosure.

The term, “alkyl” or “(un)substituted linear or branched alkyl”, as used herein, refers to the radical of saturated aliphatic groups, including straight or branched-chain alkyl groups having 25 or fewer carbon atoms in its backbone, for instance, C₁-C₂₅ for straight chain and C₃-C₂₅ for branched chain. As used herein, alkyl refers to an alkyl group having from 1 to 25 carbon atoms. Representative examples of alkyl include, but are not limited to, methyl, ethyl, n-propyl, n-butyl, n-pentyl, n-hexyl, isopropyl, sec-butyl, isobutyl, tert-butyl, isopentyl, 2-methylbutyl, 3-methylbutyl, methylundecyl, methylbiundecyl, 2-ethylhexyl, etc.

Furthermore, unless stated otherwise, the alkyl group can be unsubstituted or substituted with one or more substituents, for example, from one to four substituents, independently selected from the group consisting of halogen, hydroxy, cyano, nitro and amino. Examples of substituted alkyl include, but are not limited to hydroxymethyl, 2-chlorobutyl, trifluoromethyl and aminoethyl.

The term “(un)substituted linear or branched alkylene” as used herein refers to the radical of unsaturated double bond containing aliphatic groups, including straight or branched-chain alkylene groups having 25 or fewer carbon atoms in its backbone, for instance, C₂-C₂₅ for straight chain and C₄-C₂₅ for branched chain. The examples may be same as covered for alkyl group with one or more double bond.

The term “(un)substituted linear or branched alkynyl” as used herein refers to the radical of unsaturated triple bond containing aliphatic groups, including straight or branched-chain alkynyl groups having 25 or fewer carbon atoms in its backbone, for instance, C₂-C₂₅ for straight chain and C₄-C₂₅ for branched chain. The examples may be same as covered for alkyl group with one or more triple bond.

The term “(un)substituted aryl” as used herein refers to monocyclic or bicyclic hydrocarbon groups having 6 to 18 ring carbon atoms, wherein at least one carbocyclic ring is having a π electron system. Examples of (C₆-C₁₀) aryl ring systems include, but are not limited to, phenyl, naphthyl, anthracene, etc. Unless indicated otherwise, aryl group can be unsubstituted or substituted with one or more substituents, for example 1-4 substituents independently selected from the group consisting of halogen, (C₁₋₆)alkyl, hydroxy, cyano, nitro, —COOH, amino, alkylamino, etc.

The term “(un)substituted heteroaryl” as used herein refers to partially unsaturated or unsaturated monocyclic or bicyclic ring system containing 1 to 4 heteroatoms independently selected from the group consisting of oxygen, nitrogen and sulfur. The partially unsaturated heterocyclic ring systems contain at least one double bond, and unsaturated heterocyclic ring systems form an aromatic system containing heteroatom(s). The oxidized form of the ring nitrogen and sulfur atom contained in the heteroaryl to provide the corresponding N-oxide, S-oxide or S,S-dioxide is also encompassed in the scope of the present disclosure. Representative examples of heteroaryl include, but are not limited to, tetrahydrofuran, tetrahydrothiophene, pyrrolidine, dihydropyran, tetrahydropyran, thio-dihydropyran, thio-tetrahydropyran, piperidine, piperazine, morpholine, 1,3-oxazinane, 1,3-thiazinane, 4,5,6-tetrahydropyrimidine, 2,3-dihydrofuran, dihydrothiene, dihydropyridine, tetrahydropyridine, isoxazolidine, pyrazolidine, furan, pyrrole, thiophene, imidazole, oxazole, thiazole, triazole, tetrazole, benzofuran, indole, benzoxazole, benzothiazole, isoxazole, triazine, purine, pyridine, pyrazine, quinoline, isoquinoline, phenazine, oxadiazole, pteridine, pyridazine, quinazoline, pyrimidine, isothiazole, benzopyrazine and tetrazole. Unless stated otherwise, 5 to 10-membered heteroaryl can be unsubstituted or substituted with one or more substituents, for example, substituents independently selected from the group consisting of oxo, halogen, hydroxy, cyano, nitro, amine, (C₁-6)alkyl and COOH.

In an embodiment, the present disclosure relates to solvent free organic liquid compounds which exhibits thermally activated delayed fluorescence property (TADF) comprising the compound of Formula (I);

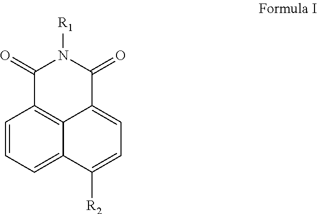

wherein,

R₁ is

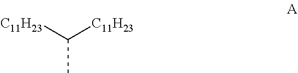

R₂ are independently selected from the group consisting of

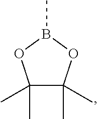

—O—R₃, —NR₄R₅, -Ph-NR₈R₉;

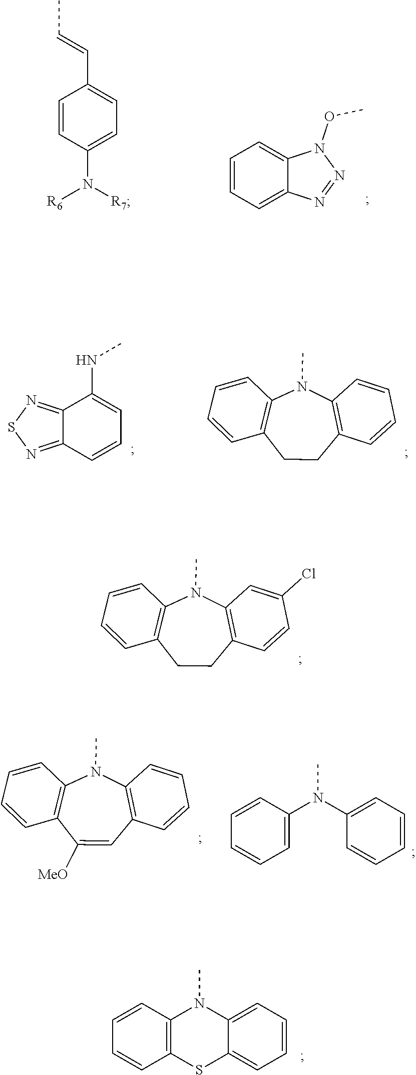

and

R₃, R₄, R₅, R₆, R₇, R₈, and R₉ are independently selected from the group consisting of (un)substituted linear or branched alkyl, (un)substituted linear or branched alkylene or (un)substituted linear or branched alkynyl, (un)substituted aryl or heteroaryl.

In another embodiment, the present disclosure relates to a solvent free organic liquid compound of formula (I) comprising:

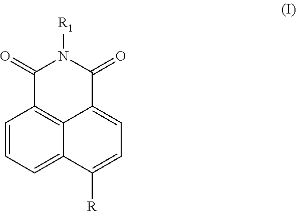

wherein, R₁ is

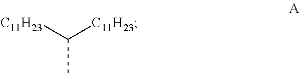

and
R₂ is selected from

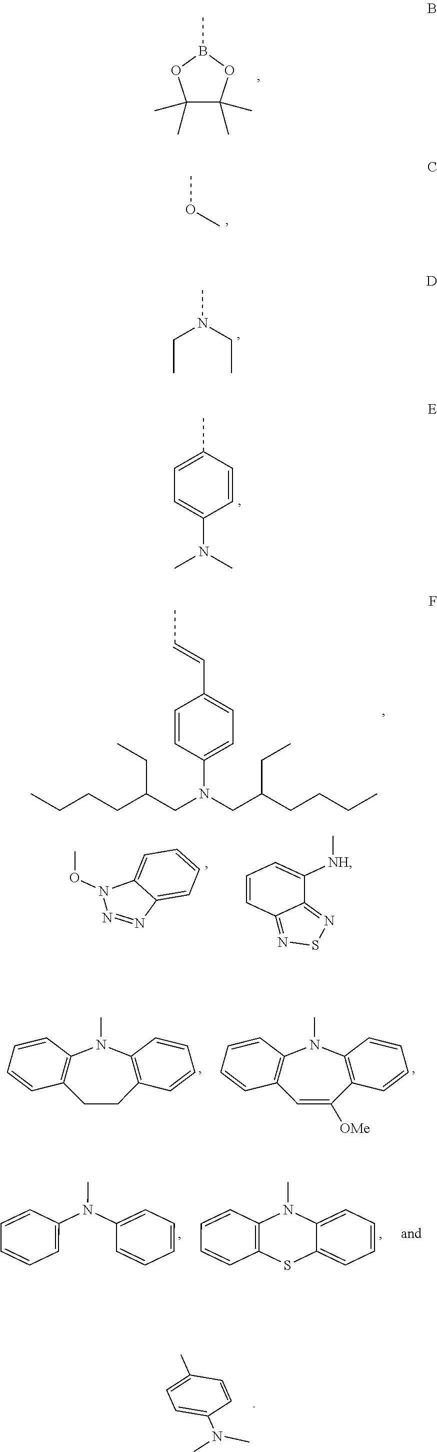

In another embodiment, the representative compounds of formula I are:

| Sr. No. | Structure | IUPAC name |
|---|---|---|
| 1. | 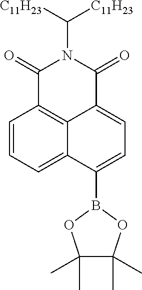 | 6-(4,4,5,5-tetramethyl-1,3,2-dioxaborolan-2-yl)-2- (tricosan-12-yl)-1H-benzo[de]isoquinoline-1,3(2H)-dione (1). |
| (1aa) | 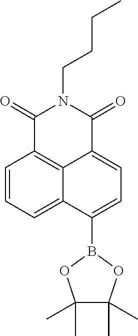 [SMILES: CCCCN1C(=O)c2cccc3c(B4OC(C)(C)C(C)(C)O4)ccc(c23)C1=O] | 2-butyl-6-(4,4,5,5-tetramethyl-1,3,2-dioxaborolan-2-yl)- 1H-benzo[de]isoquinoline-1,3(2H)-dione(1a) |
| 2. | 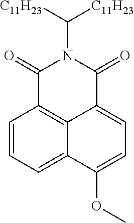 | 6-methoxy-2-(tricosan-12-yl)-1H-benzo[de]isoquinoline- 1,3(2H)-dione(2) |
| 3. | 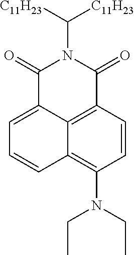 | 6-(diethylamino)-2-(tricosan-12-yl)-1H- benzo[de]isoquinoline-1,3(2H)-dione(3) |
| 4. | 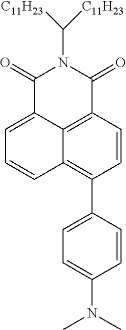 | 6-(4-(dimethylamino)phenyl)-2-(tricosan-12-yl)-1H- benzo[de]isoquinoline-1,3(2H)-dione(4) |
| 5. | 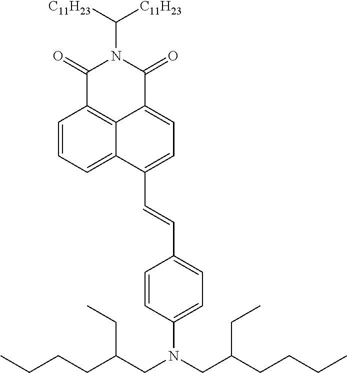 | (E)-6-(4-(bis(2-ethylhexyl)amino)styryl)-2-(tricosan-12- yl)-1H-benzo[de]isoquinoline-1,3(2H)-dione(5) |
| 6. | 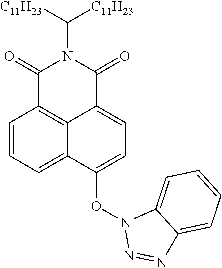 | 6-((1H-benzo[d][1,2,3]triazol-1-yl)oxy)-2-(tricosan-12- yl)-1H-benzo[de]isoquinoline-1,3(2H)-dione |
| 7. | 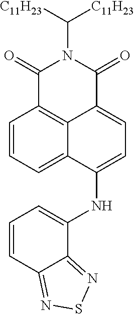 | 6-(benzo[c][1,2,5]thiadiazol-4-ylamino)-2-(tricosan-12- yl)-1H-benzo[de]isoquinoline-1,3(2H)-dione |
| 8. | 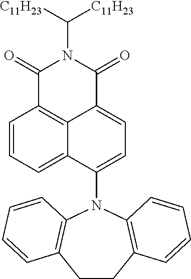 | 6-(10,11-dihydro-5H-dibenzo[b,f]azepin-5-yl)-2- (tricosan-12-yl)-1H-benzo[de]isoquinoline-1,3(2H)-dione |
| 9. | 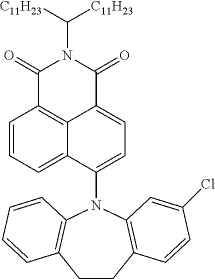 | 6-(3-chloro-10,11-dihydro-5H-dibenzo[b,f]azepin-5-yl)- 2-(tricosan-12-yl)-1H-benzo[de]isoquinoline-1,3(2H)- dione |
| 10. | 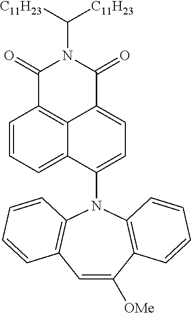 | 6-(10-methoxy-5H-dibenzo[b,f]azepin-5-yl)-2-(tricosan- 12-yl)-1H-benzo[de]isoquinoline-1,3(2H)-dione |
| 11. | 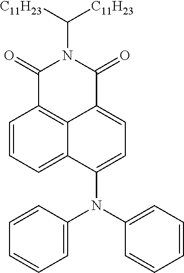 | 6-(diphenylamino)-2-(tricosan-12-yl)-1H- benzo[de]isoquinoline-1,3(2H)-dione |
| 12. | 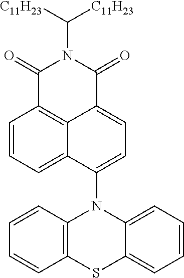 | 6-(10H-phenothiazin-10-yl)-2-(tricosan-12-yl)-1H- benzo[de]isoquinoline-1,3(2H)-dione |

In another embodiment, the present disclosure provides a method of preparation of solvent free organic liquid compounds of formula (I) comprising steps of: reacting compound (1a) with a reactant in presence of solvent, base and optional ingredients under temperature in the range of 100 to 150° C. for a time period ranging from 9-24 hrs to obtain a compound of formula (I),

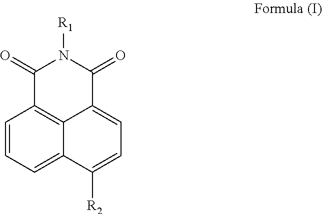

wherein one of optional ingredient is catalyst;

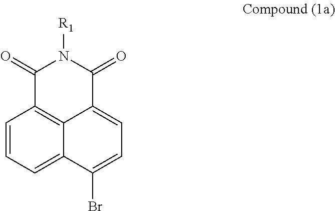

wherein, R₁ is

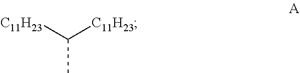

R₂ is independently selected from the group consisting of

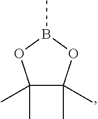

—O—R₃, —NR₄R₅, -Ph-NR₈R₉;

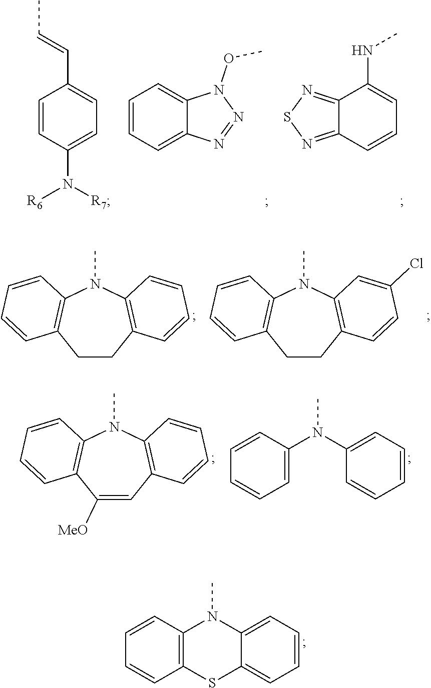

and

R₃, R₄, R₅, R₆, R₇, R₈, and R₉ are independently selected from (un)substituted linear or branched alkyl, (un)substituted linear or branched alkylene or (un)substituted linear or branched alkynyl, (un)substituted aryl or heteroaryl.

In another embodiment, the reactant is selected from the group consisting of

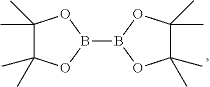

### MeOH,

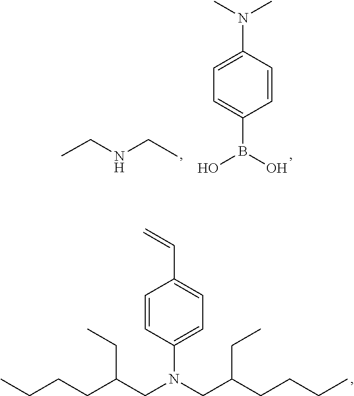

1-hydroxybenzotriazole, tri-tert-butylphosphonium tetrafluoroborate, 10,11-dihydro-5H-dibenzo[b,f]azepine, 3-chloro-10,11-dihydro-5H-dibenzo[b,f]azepine, 10-methoxy-5H-dibenzo[b,f]azepine, Diphenylamine, and 10H-phenothiazine.

In yet another embodiment, the catalyst being one of optional ingredient is selected from a group consisting of Pd(dppf)Cl₂, Pd(PPh₃)₄, Pd(OAc)₂, Pd₂(dba)₃, and Pd(dba)₂.

In yet another embodiment, further optional ingredient is selected from tris(o-tolyl)phosphine, tri-tert-butylphosphonium tetrafluoroborate, N,N-bis(2-ethylhexyl)-4-vinylaniline, benzo[c][1,2,5]thiadiazol-4-amine, BINAP and CuSO₄·5H₂O.

In yet another embodiment, the base is selected from a group consisting of KOAc, NaOAc, triethylamine, sodium-t-butoxide, Cs₂CO₃, K₂CO₃, NaOC(CH₃)₃ and KOC(CH₃)₃.

In yet another embodiment, the solvent is selected from a group consisting of 1,4-dioxane, MeOH, toluene and DMF.

In an embodiment, the present disclosure discloses, the compound (1a) is reacted with

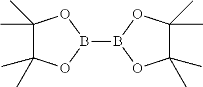

in presence of a catalyst, base, and solvent at reflux for a time period in the range of 14 to 18 hrs to form the compound of formula (I) or a compound (1).

In an embodiment, the present disclosure discloses the catalyst selected from a group consisting of Pd(dppf)Cl₂, Pd(PPh₃)₄, Pd(OAc)₂, Pd₂ (dba)₃ and Pd(dba)₂, base selected from a group consisting of KOAc, NaOAc, K₂CO₃, NaOC(CH₃)₃ and KOC(CH₃)₃ and solvent selected from a group consisting of 1,4-dioxane, toluene and DMF. Preferably, the catalyst, base and solvent are pd (dppf)Cl₂, KOAc and 1,4-dioxane respectively.

In an embodiment, the present disclosure discloses the compound (1a) is reacted with MeOH in presence of a base at reflux for a period in the range of 10-14 hrs to obtain the compound of formula (I) or a compound (2).

In an embodiment, the present disclosure discloses, the base selected from a group consisting of KOAc, NaOAc, K₂CO₃, NaOC(CH₃)₃, KOC(CH₃)₃. Preferably the base is K₂CO₃.

In an embodiment, the present disclosure discloses, the compound (1a) is reacted with

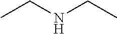

in presence of CuSO₄·5H₂O and solvent to form the compound of formula (I) or a compound (3).

In an embodiment, the present disclosure discloses the solvent selected from a group consisting of 1,4-dioxane, toluene and DMF. Preferably the solvent is DMF.

In an embodiment, the present disclosure discloses the compound (1a) is reacted with

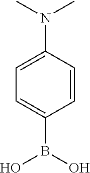

in present of a catalyst, base and solvent at a temperature in the range of 110-130° C. for a period in the range of 20-28 hrs to form the compound of formula (I) or a compound (4).

In an embodiment, the present disclosure discloses the catalyst selected from a group consisting of Pd(dppf)Cl₂, Pd(PPh₃)₄, Pd(OAc)₂, Pd₂ (dba)₃ and Pd(dba)₂, base selected from a group consisting of KOAc, NaOAc, K₂CO₃, NaOC(CH₃)₃ and KOC(CH₃)₃ and solvent selected from a group consisting of 1,4-dioxane, toluene and DMF. Preferably, the catalyst, base and solvent are Pd(PPh₃)₄, K₂CO₃ and toluene respectively.

In an embodiment, the present disclosure discloses the compound (1a) is reacted with

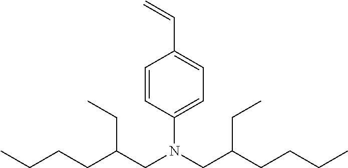 [SMILES: C=Cc1ccc(N(CC(CC)CCCC)CC(CC)CCCC)cc1]

in present of a catalyst and solvent at a temperature in the range of 110-130° C. to form the compound of formula (I) or a compound (5).

In an embodiment, the present disclosure discloses the catalyst selected from a group consisting of Pd(dppf)Cl₂, Pd(PPh₃)₄, Pd(OAc)₂, Pd₂ (dba)₃ and Pd(dba)₂, base selected from a group consisting of KOAc, NaOAc, K₂CO₃, NaOC(CH₃)₃ and KOC(CH₃)₃ and solvent selected from a group consisting of 1,4-dioxane, toluene and DMF. Preferably, the catalyst and solvent are P (o-toll)₃, Pd₂ (dba)₃ and TEA and DMF respectively.

Another embodiment, the present disclosure discloses a composition comprising the solvent free organic liquid compound of Formula (I) or Formula (II) and formulating agents/excipients, exhibiting thermally activated delayed fluorescence (TADF) with high luminescence quantum yields of 60-80% and lifetimes of 8-45 μs.

In another embodiment, the present disclosure provides solvent free organic liquid which exhibits thermally activated delayed fluorescence property (TADF) comprising the compound of Formula (II),

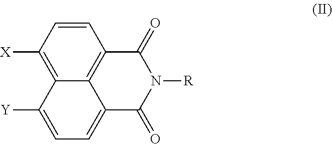

wherein ‘X’ and “Y” are independently selected from hydrogen,

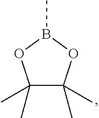

—O—R₃, —NR₄R₅, -Ph-NR₈R₉;

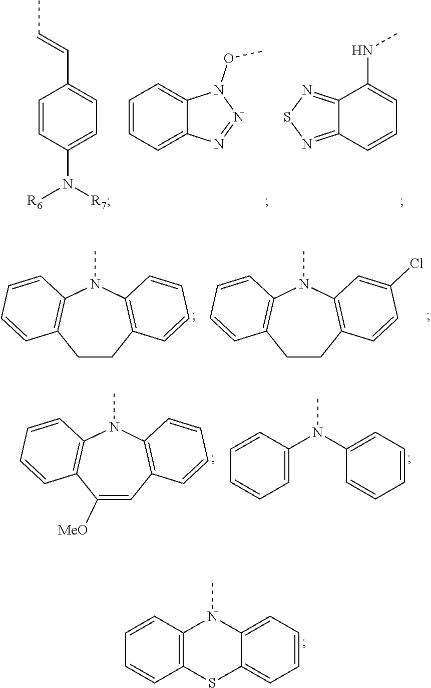

and

R₃, R₄, R₅, R₆, R₇, R₈, and R₉ are independently selected from (un)substituted linear or branched alkyl, (un)substituted linear or branched alkylene or (un)substituted linear or branched alkynyl, (un)substituted aryl or heteroaryl and;

‘R’ is selected independently from (un)substituted linear or branched alkyl, (un)substituted linear or branched alkylene,

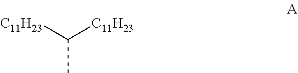

or (un)substituted linear or branched alkynyl.

In another embodiment, the R group of formula (II) is octyl or 2-ethyl hexyl; X is hydrogen or substituted aryl selected from Ph-NR₈R₉ (e.g. para-dimethylaminophenyl group); and Y is hydrogen or substituted aryl selected from Ph-NR₈R₉ (e.g. para-dimethylaminophenyl group).

The representative compound of formula (II) is:

| | | |
|---|---|---|
| 1. | 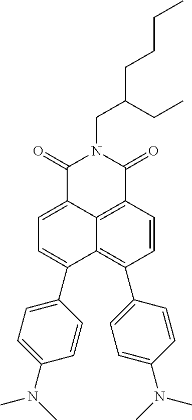 | 6,7-bis(4-(dimethyl amino)phenyl)-2-(2- ethylhexyl)-1H-benzo[de]isoquinoline- 1,3(2H)-dione |

In another embodiment, the compounds of Formula (I) and Formula (II) may be in solid form.

In yet another embodiment, the present disclosure provides the process for preparation of the compounds of formula (I).

Accordingly, the compound 6-(4,4,5,5-tetramethyl-1,3,2-dioxaborolan-2-yl)-2-(tricosan-12-yl)-1H-benzo[de]isoquinoline-1,3 (2H)-dione of Formula (1) is prepared by the process comprising; Miyaura borylation of 6-bromo-2-(tricosan-12-yl)-1H benzo[de]isoquinoline-1,3 (2H)-dione with bis(pinacolato)diboron in presence of anhydrous solvent followed by reaction with dichloro[1,1′-bis(diphenylphospheno)-ferrocene]palladium (II) to obtain 6-(4,4,5,5-tetramethyl-1,3,2-dioxaborolan-2-yl)-2-(tricosan-12-yl)-1H-benzo[de]isoquinoline-1,3 (2H)-dione (1).

The compound 6-methoxy-2-(tricosan-12-yl)-1H-benzo[de]isoquinoline-1,3 (2H)-dione of formula (2) is prepared by the process comprising; dissolving 6-bromo-2-(tricosan-12-yl)-1H-benzo[de]isoquinoline-1,3 (2H)-dione and base in anhydrous methanol and heating the mixture at reflux to obtain 6-methoxy-2-(tricosan-12-yl)-1H-benzo[de]isoquinoline-1,3 (2H)-dione (2).

The compound 6-(diethylamino)-2-(tricosan-12-yl)-1H-benzo[de]isoquinoline-1,3 (2H)-dione of formula (3) is prepared by process comprising; mixing 6-bromo-2-(tricosan-12-yl)-1H-benzo[de]isoquinoline-1,3 (2H)-dione, diethylamine, base, CuSO₄5H₂O as catalyst in solvent; and stirring the above mixture at about 90° C. for 12-14 hours to obtain 6-(diethylamino)-2-(tricosan-12-yl)-1H-benzo[de]isoquinoline-1,3 (2H)-dione (3).

The compound 6-(4-(dimethylamino)phenyl)-2-(tricosan-12-yl)-1H-benzo[de]isoquinoline-1,3 (2H)-dione of formula (4) is prepared by the process comprising; dissolving 6-bromo-2-(tricosan-12-yl)-1H-benzo[de]isoquinoline-1,3 (2H)-dione and 4-(N,N-dimethylamino)phenylboronic acid in the solvent under inert atmosphere followed by addition of the base and tetrakis(triphenylphosphine)palladium(0); heating the mixture under inert atmosphere to yield 6-(4-(dimethylamino)phenyl)-2-(tricosan-12-yl)-1H-benzo[de]isoquinoline-1,3 (2H)-dione (4).

The compound (E)-6-(4-(bis(2-ethylhexyl)amino) styryl)-2-(tricosan-12-yl)-1H-benzo[de]isoquinoline-1,3 (2H)-dione of formula (5) is prepared by the process comprising; heating the mixture of aniline, 2-ethylhexyl bromide in base and the solvent at about 160° C. to obtain the product N,N-bis(2-ethylhexyl) aniline, adding N,N-bis(2-ethylhexyl) aniline to the cooled solution of N,N-dimethylformamide, phosphorous oxytrichloride maintained under inert atmosphere; and heating the above mixture to obtain 4-(bis(2-ethylhexyl)amino)benzaldehyde; preparing the solution of Methyl triphenyl-phosphonium bromide in THF, cooling to −78° C., followed by addition of solution of n-BuLi under nitrogen, stirring the reaction mixture at −78° C. and then warming to room temperature; and adding dropwise the solution of 4-(bis(2-ethylhexyl)amino) benzaldehyde, stirring to obtain N,N-bis(2-ethylhexyl)-4-vinylaniline; heating the mixture of 6-bromo-2-(tricosan-12-yl)-1H-benzo[de]isoquinoline-1,3 (2H)-dione, Pd(OAc)₂, tris-(ortho-tolyl)phosphine, base and N,N-bis(2-ethylhexyl)-4-vinylaniline to yield (E)-6-(4-(bis(2-ethylhexyl)amino) styryl)-2-(tricosan-12-yl)-1H-benzo[de]isoquinoline-1,3 (2H)-dione (5).

The compound 6-((1H-benzo[d][1,2,3]triazol-1-yl)oxy)-2-(tricosan-12-yl)-1H-benzo[de]isoquinoline-1,3 (2H)-dione (6) is prepared by the process comprising; dissolving 6-bromo-2-(tricosan-12-yl)-1H-benzo[de]isoquinoline-1,3 (2H)-dione and 1-hydroxybenzitriazole in the solvent under inert atmosphere; heating the mixture under inert atmosphere to yield 6-((1H-benzo[d][1,2,3]triazol-1-yl)oxy)-2-(tricosan-12-yl)-1H-benzo[de]isoquinoline-1,3 (2H)-dione. 1,3 (2H)-dione (6)

The compound 6-(benzo[c][1,2,5]thiadiazol-4-ylamino)-2-(tricosan-12-yl)-1H-benzo[de]isoquinoline-1,3 (2H)-dione (7) is prepared by the process comprising; dissolving 6-bromo-2-(tricosan-12-yl)-1H-benzo[de]isoquinoline-1,3 (2H)-dione, benzo[c][1,2,5]thiadiazol-4-amine, Pd₂ (dba)₃, tri-tert-butylphosphonium tetrafluoroborate, base in the solvent under inert atmosphere to yield 6-(benzo[c][1,2,5]thiadiazol-4-ylamino)-2-(tricosan-12-yl)-1H-benzo[de]isoquinoline-1,3 (2H)-dione.

The compound 6-(10,11-dihydro-5H-dibenzo[b,f]azepin-5-yl)-2-(tricosan-12-yl)-1H-benzo[de]isoquinoline-1,3 (2H)-dione (8) is prepared by the process comprising; dissolving 6-bromo-2-(tricosan-12-yl)-1H-benzo[de]isoquinoline-1,3 (2H)-dione and 10,11-dihydro-5H-dibenzo[b,f]azepine, tri(o-tolyl)phosphine in the solvent under inert atmosphere followed by addition of the base and Bis(dibenzylideneacetone)palladium(0); heating the mixture under inert atmosphere to yield 6-(10,11-dihydro-5H-dibenzo[b,f]azepin-5-yl)-2-(tricosan-12-yl)-1H-benzo[de]isoquinoline-1,3 (2H)-dione.

The compound 6-(3-chloro-10,11-dihydro-5H-dibenzo[b,f]azepin-5-yl)-2-(tricosan-12-yl)-1H-benzo[de]isoquinoline-1,3 (2H)-dione (9) is prepared by the process comprising: dissolving 6-bromo-2-(tricosan-12-yl)-1H-benzo[de]isoquinoline-1,3 (2H)-dione and 3-chloro-10,11-dihydro-5H-dibenzo[b,f]azepine, tri(o-tolyl)phosphine in the solvent under inert atmosphere followed by addition of the base and Bis(dibenzylideneacetone)palladium(0); heating the mixture under inert atmosphere to yield 6-(3-chloro-10,11-dihydro-5H-dibenzo[b,f]azepin-5-yl)-2-(tricosan-12-yl)-1H-benzo[de]isoquinoline-1,3 (2H)-dione.

The compound 6-(10-methoxy-5H-dibenzo[b,f]azepin-5-yl)-2-(tricosan-12-yl)-1H-benzo[de]isoquinoline-1,3 (2H)-dione (10) is prepared by the process comprising: dissolving 6-bromo-2-(tricosan-12-yl)-1H-benzo[de]isoquinoline-1,3 (2H)-dione and 10-methoxy-5H-dibenzo[b,f]azepine, tri(o-tolyl)phosphine in the solvent under inert atmosphere followed by addition of the base and Bis(dibenzylideneacetone)palladium(0); heating the mixture under inert atmosphere to 6-(10-methoxy-5H-dibenzo[b,f]azepin-5-yl)-2-(tricosan-12-yl)-1H-benzo[de]isoquinoline-1,3 (2H)-dione.

The compound 6-(diphenylamino)-2-(tricosan-12-yl)-1H-benzo[de]isoquinoline-1,3 (2H)-dione (11) is prepared by the process comprising: dissolving 6-bromo-2-(tricosan-12-yl)-1H-benzo[de]isoquinoline-1,3 (2H)-dione and diphenylamine, tri-tert-butylphosphonium tetrafluoroborate in the solvent under inert atmosphere followed by addition of the base and palladium acetate (II); and heating the mixture under inert atmosphere to 6-(diphenylamino)-2-(tricosan-12-yl)-1H-benzo[de]isoquinoline-1,3 (2H)-dione.

The compound 6-(10H-phenothiazin-10-yl)-2-(tricosan-12-yl)-1H-benzo[de]isoquinoline-1,3 (2H)-dione (12) is prepared by the process comprising: dissolving 6-bromo-2-(tricosan-12-yl)-1H-benzo[de]isoquinoline-1,3 (2H)-dione and 10H-phenothiazine, BINAP in the solvent under inert atmosphere followed by addition of the base and Pd₂ (dba)₃; heating the mixture under inert atmosphere to 6-(10H-phenothiazin-10-yl)-2-(tricosan-12-yl)-1H-benzo[de]isoquinoline-1,3 (2H)-dione.

The compound 6,7-bis(4-(dimethylamino)phenyl)-2-(2-ethylhexyl)-1H-benzo[de]isoquinoline-1,3 (2H)-dione (13) is prepared by the process comprising: dissolving 6,7-dibromo-2-(2-ethylhexyl)-1H-benzo[de]isoquinoline-1,3 (2H)-dione and (4-(dimethylamino)phenyl) boronic acid, in the solvent under inert atmosphere followed by addition of the base and tetrakis(triphenylphosphine)palladium(0); heating the mixture under inert atmosphere to 6,7-bis(4-(dimethylamino)phenyl)-2-(2-ethylhexyl)-1H-benzo[de]isoquinoline-1,3 (2H)-dione.

In yet another embodiment, the present disclosure relates to the formulation of solvent free organic liquid that exhibits thermally activated delayed fluorescence property (TADF) of Formula (I) or Formula (II) together with suitable formulating agents.

In another embodiment, the solvent-free organic liquid of Formula (I) exhibit blue to red TADF with high luminescence quantum yields of 60-80% and lifetimes of 8-45 μs.

The solvent-free organic compounds of Formula (I) or Formula (II) of present disclosure are free flowing, viscous and amorphous in nature.

In an embodiment, the long branched alkyl chains present in the solvent-free organic compounds of Formula (I) or Formula (II) of the present disclosure diminishes the concentration-induced quenching that originates from π-π stacking and offers an intense monomer emission in bulk.

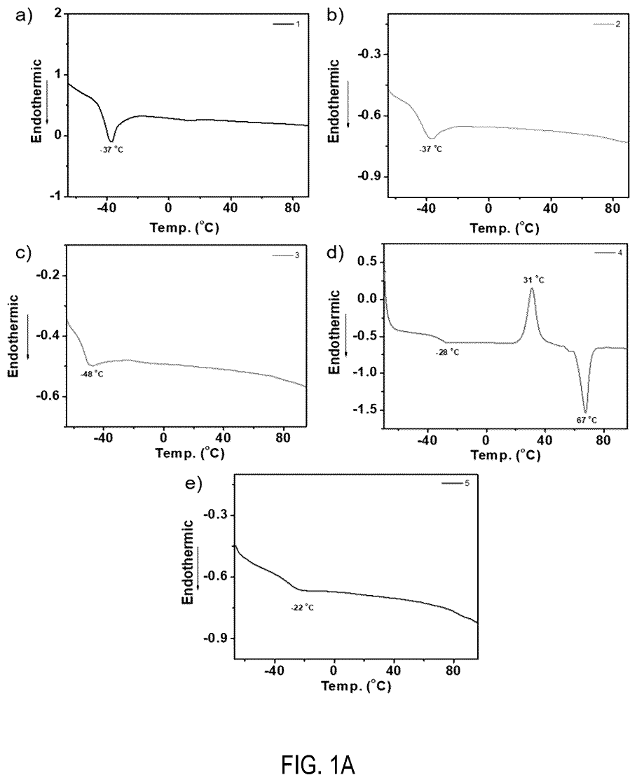

In an embodiment, the compound of formula 1 to 5 was characterized by DSC, TGA. The compounds exhibited high stability with a very high decomposition temperature (T_{d}) in TGA. The compound (1) exhibited excellent thermal stability with 5% weight loss at 284° C. FIG. 1A showed the DSC thermograms of compounds 1-5 at a scanning rate of 10° C./min, showing the T_{goffset} values and FIG. 1B showed TGA of compounds 1-5 at a scanning rate of 10° C./min, showing the T_{goffset} values. In conclusion, the free-flowing, viscous and amorphous nature and thermal stability of 1-5 was characterized by differential scanning calorimetry, thermogravimetric analysis.

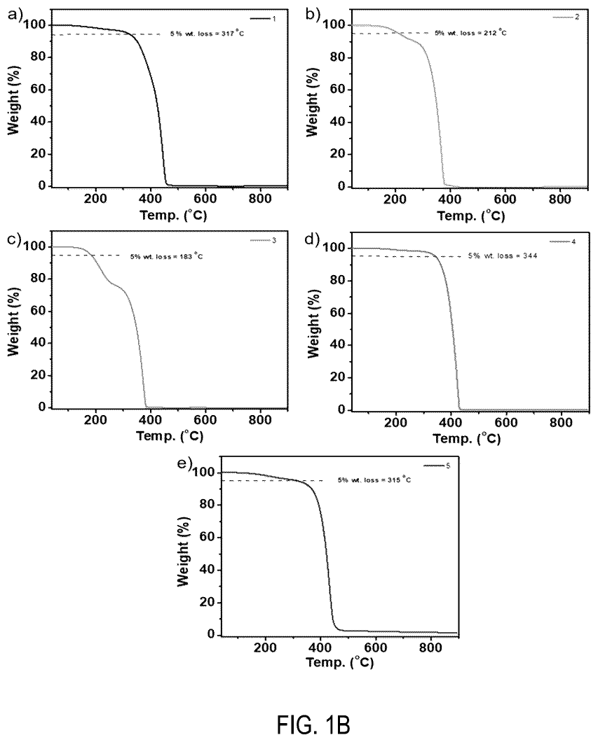

In yet another embodiment, the solvent-free organic compounds of Formula (I) or Formula (II) exhibit viscosity of about 1.87 Pas at 1 rads-1 in the liquid state due to the highly dynamic molecular interactions as demonstrated for compound (1) (FIG. 2). The liquids of the present disclosure possess a certain degree of viscoelasticity which allows the compounds to be used as paints that can be applied on a large area and with enhanced processability as compared to conventional crystalline emitters.

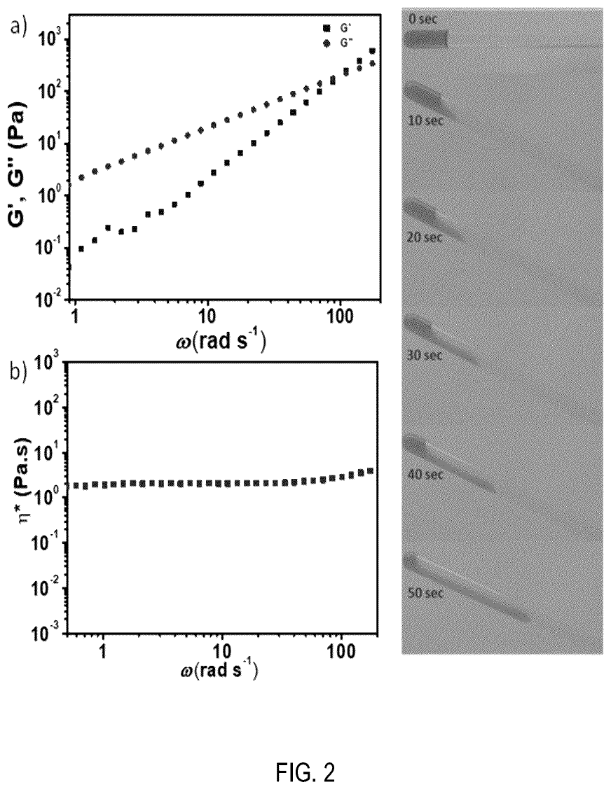

In an embodiment, FIG. 3 showed Fluorescence lifetime decay profiles of 1-5 in a) in MTHF solution (C=1×10⁻⁵ M) and b) in neat state at 298 K (λₑₓ=374 nm, λₘₒₙ=420 nm for 1,420 nm for 2, 524 nm for 3, 564 nm for 4, 620 nm for 5). Fluorescence lifetime measurements of 1-5 in solution and neat state showed different exponential decays with major lifetimes of 1-5 in ns.

In an embodiment, Emission lifetime of 1-5 in MTHF solution (C=10⁻⁵M) and neat state at 298 K (Table 3).

In an embodiment, TADF life time of 1 is found as bi-exponential decay with lifetime of 7.9 μs.

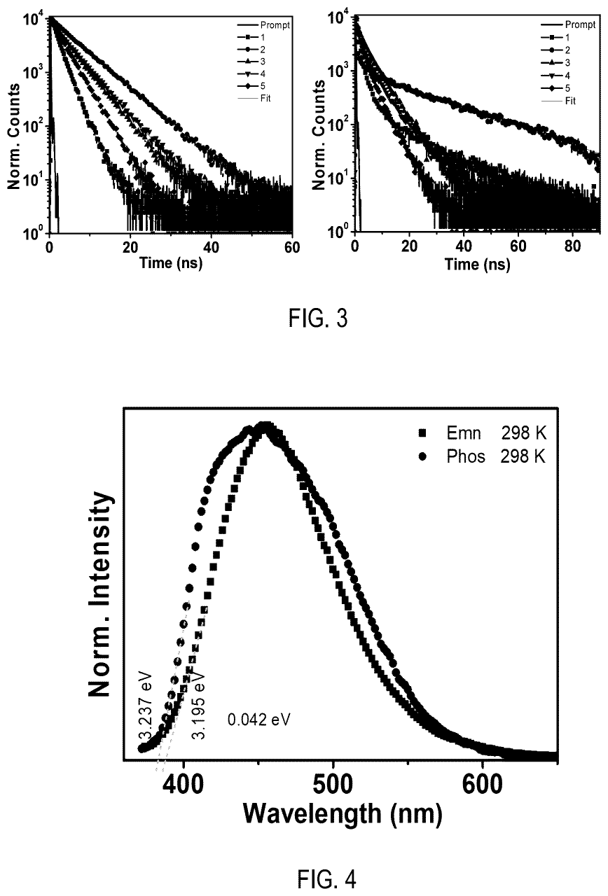

Further, the similarity in shape and significant overlap between the fluorescence and phosphorescence spectra confirmed the minimal singlet-triplet energy gap (AEST) of compound (1) as shown in FIG. 4. The steady-state emissions and phosphorescence spectrum at 298 K look identical. It validates the support of molecular design and liquid features in imparting TADF property for 1-5.

In an embodiment, the TADF property and the lifetime of solvent-free organic liquid of Formula (I) or Formula (II) of present disclosure increase exponentially with increase in temperature (Table 3). The intensities of emission spectra increased upon increasing the temperature and it demonstrates the origin of a typical TADF feature. The compound (1) shows an increase in lifetime upon increasing the temperature from 77K to 298K. The bi-exponential decay was retained upon increasing the temperature from 77 K (0.45 μs) to 298 K (7.90 μs). This observation unambiguously confirms the TADF property, which indicates the existence of a thermal activation energy barrier for TADF, and thus more favorable reverse intersystem crossing (RISC) of excitons from triplet to singlet excited state by gradual warming.

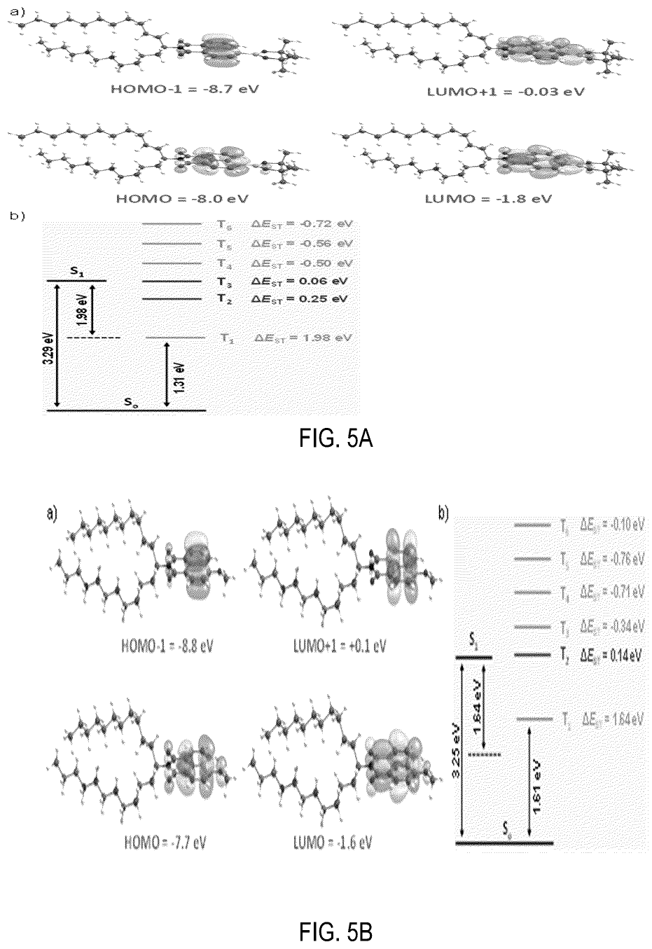

Further, a series of TD-DFT computations were carried out for 1-5 by using Gaussian 09 program. The HOMO and LUMO energy level diagrams (FIGS. 5A-5E) of the compounds 1-5 indicate that the compounds have well-matched singlet and triplet energy states which helps in the efficient reverse intersystem crossing from triplet to the singlet. On examining ΔE_{ST}, triplet states are nearly degenerate with the first singlet excited state (S₁) having ΔE_{ST} of 0.14-0.60 eV, respectively.

The SOL compounds of the present disclosure showed delayed fluorescence of TADF nature in the neat liquid state, as demonstrated by the lifetime and spectral pattern and 298 and 77 K (tables below), and exhibited good emission quantum yield, (QY) depicted in table 3 below.

Tunable emission hybrid liquids of 1 were developed by mixing varying amounts of 3 and 4, compound 2 with 3, 4 and 5. Molecule 1 and 2 acted as a good donor to exhibit efficient ET with dopants to deliver tunable emission colors upon exciting at 365 nm.

The nonpolar liquid matrix of 1 and 2 showed excellent compatibility with the dopants without making any phase separation, which is not feasible with the thin films of the solid counterparts. Interestingly, an enhanced ET supported by small ΔE_{ST} provides an additional relaxation path for TADF donors and thus leads to a reduced exciton concentration and lifetime.

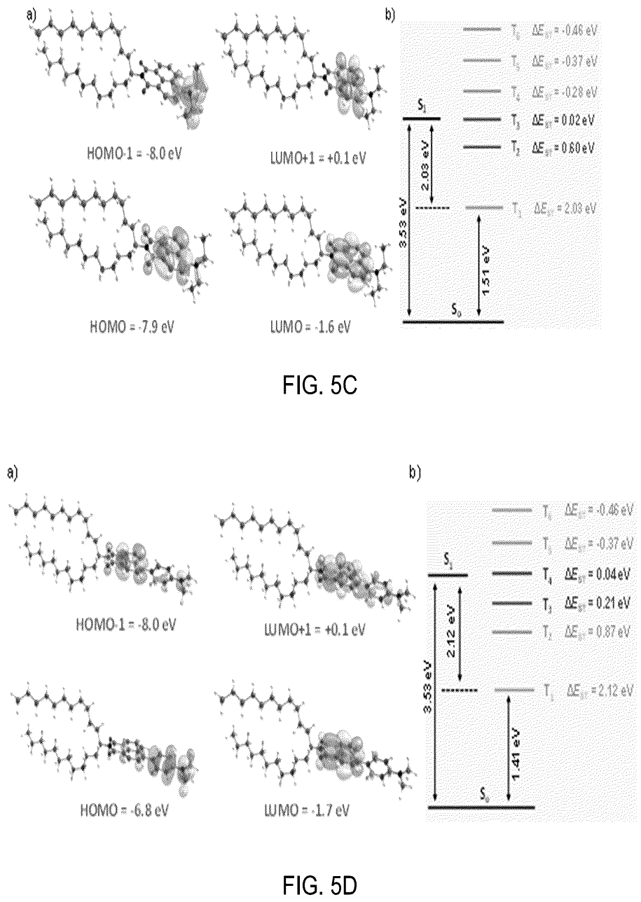

In an embodiment, FIG. 6 showed the Comparison of the emission spectra in of 1-5 in neat state at 298 K and 77 K (λₑₓ=355 nm for 1, λₑₓ=360 nm for 2, λₑₓ=424 nm for 3, λₑₓ=434 nm for 4, λₑₓ=480 nm for 5). The similarity in shape and significant overlap between steady-state emissions spectra at 298 K and 77 K looks similar, confirmed minimal S₁-T₁ energy gap of 1-5.

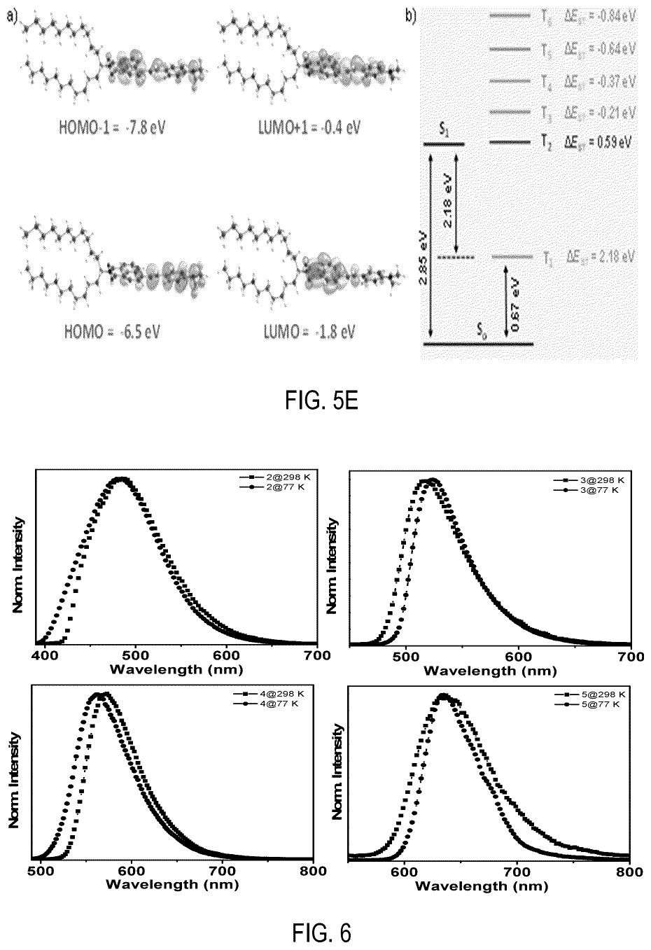

In an embodiment, FIG. 7 showed the Spectra overlap integral of emission and absorption of a)₁ and 3, b)₁ and 4, c)₁ and 4, d)₂ and 3, e)₂ and 4, f)₂ and 5, respectively, in MTHF solution. SOLs 1-5 have been chosen for energy transfer (ET) studies due to the significant overlap between the dopants' (3-5) absorption spectra with emission of the donors 1 and 2.

In an embodiment, 1 wt % of the SOL of the present disclosure were mixed with polylactic acid (PLA) for 3D-printing applications. SOLs individually and in combinations have been used to make a cube having sides with blue (1), cyan (2), green (3), orange (4), and red (5) along with white (1, 3, and 5) emissions under UV light (365 nm) excitation. The high processability of SOLs exhibited uniform distribution in the PLA matrix and provided no opportunity for phase separation.

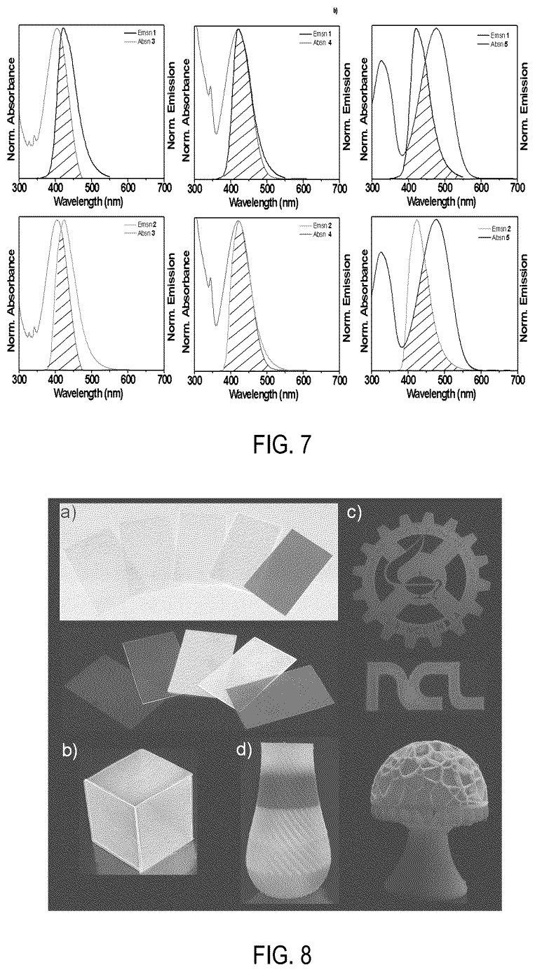

In an embodiment, 1 FIG. 8 a) showed photograph of the SOL (1-5)-PLA hybrid sheets under visible (top) and UV (365 nm) (bottom) lights, and b) a cube with different emission colors (1-5), including white (2+5) (1:0.002) made out of hybrid sheets under UV (365 nm) light. Photographs of the 3D printed objects using PLA-TADF SOL (1 wt %) hybrids under UV light (365 nm). c) CSIR and NCL logo using 1-PLA and d) colorful flower vase (2-5 and 2+5) and mushroom lamp (2, 5). The liquid matrix of SOLs allowed facile mixing with polylactic acid (PLA), a bio-based polymer, to yield solid filament materials ideal for 3D printing applications. We have optimized the minimum amount of SOL as 1 wt % for satisfactory luminescence features with excellent quantum yields for thin films. The photographs of the SOL doped (1 wt %) thin films under UV light (365 nm) show similar emission features as of the neat SOLs, and it points to the excellent dispersibility of TADF SOLs in the PLA matrix. SOLs exhibited excellent thermal stability and processability when melt compounded with thermoplastic polymers such as PLA, rendering their uniform distribution in the PLA matrix.

The compounds of Formula (I) and Formula (II) showed varying emission colors through structural modification. The host-guest combination of TADF liquid emitters exhibited ET-assisted tunable emission. A cascade energy transfer between the blue, green, and red-emitting TADF liquids demonstrated a white light-emitting hybrid liquid. The capability of the TDAF liquids to form a large-area thin luminescent film, and tunable emission by doping enables TADF liquids to exhibit their potentiality are in lighting and display applications. Further, high quantum yield and uniform dispersibility of the liquid hybrids of the present disclosure enabled very low loading of the emitters (1 wt %) to deliver superior multicolor light emission upon single wavelength excitation for the 3D printed objects.

### EXAMPLES

### Example 1

### Synthesis of 6-(4,4,5,5-tetramethyl-1,3,2-dioxaborolan-2-yl)-2-(tricosan-12-yl)-1H-benzo[de]isoquinoline-1,3 (2H)-dione (1)

A mixture of 6-bromo-2-(tricosan-12-yl)-1H-benzo de]isoquinoline-1,3 (2H)-dione (2 gm, 3.34 mmol), bis(pinacolato)diboron (B₂pin₂) (1.01 μm, 4.01 mmol), anhydrous potassium acetate (393.42 mg, 4.01 mmol) was weighed and taken in a two neck flask with anhydrous 1,4-dioxane (50 mL). This flask was degassed by repeated freeze-pump-thaw cycles. After this, dichloro[1,1′-bis(diphenylphospheno)-ferrocene]palladium (II) (245.10 mg, 0.33 mmol) was added to the reaction mixture and kept for reflux at 110° C. under argon for 12 hours. The progress of the reaction was monitored by TLC (n-hexane-CH₂Cl₂). The crude mixture was filtered through celite and washed with CH₂Cl₂. After the removal of CH₂Cl₂ on a rotary evaporator, the resulting residue was purified by silica column chromatography (100-200 mesh) using n-hexane: CH₂Cl₂ (50:50) (R_{f}=0.3) as eluent to yield 1 (1.38 g, 64%) as a brown colored liquid. ¹H NMR (400 MHz, CDCl₃), δ (TMS, ppm): 9.13 (d, J=7.93, 1H), 8.58 (d, J=7.93, 2H), 8.35 (d, J=8.55, 1H), 7.78 (t, J=8.55, 1H), 5.16 (m, 1H), 2.25 (m, 2H), 1.86 (m, 2H), 1.45 (s, 12H), 1.19 (s, 36H), 0.86 (t, J=6.71, 6H).

### Example 2

### Synthesis of 2-butyl-6-(4,4,5,5-tetramethyl-1,3,2-dioxaborolan-2-yl)-1H-benzo[de]isoquinoline-1,3 (2H)-dione (1aa)

A mixture of 6-bromo-2-butyl-1H-benzo[de]isoquinoline-1,3 (2H)-dione (2 μm, 6.02 mmol), bis(pinacolato)diboron (B₂pin₂) (1.83 μm, 7.22 mmol), anhydrous potassium acetate (709.04 mg, 7.22 mmol) was weighed and taken in a two neck flask with anhydrous 1,4-dioxane (50 mL). This flask was degassed by repeated freeze-pump-thaw cycles. After this, dichloro[1,1′-bis(diphenylphospheno)-ferrocene]palladium (II) (441.74 mg, 0.60 mmol) was added to the reaction mixture and kept for reflux at 110° C. under argon for 12 hours. The progress of the reaction was monitored by TLC (n-hexane-CH₂Cl₂). The crude mixture was filtered through celite and washed with CH₂Cl₂. After the removal of CH₂Cl₂ on a rotary evaporator, the resulting residue was purified by silica column chromatography (100-200 mesh) using n-hexane: CH₂Cl₂ (75:25) (R_{f}=0.6) as eluent to yield 1aa (1.46 g, 51%) as a pale yellow crystals. ¹H NMR (400 MHZ, CDCl₃), δ (TMS, ppm): 9.06 (d, J=8.16, 1H), 8.54 (t, J=7.32, 2H), 8.28 (d, J=6.48, 1H), 7.75 (d, J=8.73, 1H), 4.16 (t, J=7.51, 2H), 1.72 (m, 2H), 1.45 (s, 12H), 1.24 (m, 2H), 0.97 (t, J=6.54, 3H).

### Example 3

### Synthesis of 6-methoxy-2-(tricosan-12-yl)-1H-benzo[de]iso-quinoline-1,3 (2H)-dione (2)

Compound NMIBr 6-bromo-2-(tricosan-12-yl)-1H-benzo[de]isoquinoline-1,3 (2H)-dione (665 mg, 2.0 mmol, 1.0 eq) and K₂CO₃ (1.40 g, 10.0 mmol, 5.0 eq) were dissolved in 30 mL anhydrous MeOH. The mixture was heated to reflux and stirred overnight. After the total consumption of compound 2 (monitored by TLC), the reaction was cooled down. The precipitated solid was filtered and washed with distilled water for several time. ¹H NMR (400 MHZ, CDCl₃), δ (TMS, ppm): 8.57-8.64 (m, 1H), 8.54 (dd, J=8.4, 1.1 Hz, 2H), 7.70 (dd, J=8.2, 7.4 Hz, 1H), 7.05 (d, J=8.3 Hz, 1H), 5.12-5.21 (m, 1H), 4.13 (s, 3H), 2.18-2.28 (m, 2H), 1.77-1.87 (m, 2H), 1.17-1.35 (m, 36H), 0.86 ppm (t, J=6.9 Hz, 6H).

### Example 4

### Synthesis of 6-(diethylamino)-2-(tricosan-12-yl)-1H-benzo[de]isoquinoline-1,3 (2H)-dione (3)

Compound 6-bromo-2-(tricosan-12-yl)-1H-benzo[de]isoquinoline-1,3 (2H)-dione (0.66 g, 2.0 mmol), diethylamine (3 mL, 29.1 mmol) and CuSO₄5H₂O (0.01 g, as catalyst) in DMF (5 mL) were stirred at 90° C. for 12 h. The solvent was evaporated under reduced pressure, and the crude product was purified by column chromatography on silica to afford 3 as a yellow oil (0.24 g, 38.2%). ¹H NMR (400 MHz, CDCl₃), δ (TMS, ppm): 8.48-8.59 (m, 2H), 8.45 (d, J=8.4 Hz, 1H), 7.66 (t, J=7.8 Hz, 1H), 7.22 (d, J=8.4 Hz, 1H), 5.16 (br. s, 1H), 3.41 (q, J=7.0 Hz, 4H), 2.18-2.29 (m, 2H), 1.76-1.87 (m, 2H), 1.19-1.35 (m, 36H), 1.18-1.16 (m, 6H), 0.86 ppm (t, J=7.1 Hz, 6H).

### Example 5

### Synthesis of 6-(4-(dimethylamino)phenyl)-2-(tricosan-12-yl)-1H-benzo[de]isoquinoline-1,3 (2H)-dione (4)

A sealed tube was charged with 6-bromo-2-(tricosan-12-yl)-1H-benzo[de]isoquinoline-1,3 (2H)-dione (500 mg, 1 eq.) and 4-(N,N-dimethylamino)phenylboronic acid (207 mg, 1.5 eq.). Both the compounds dissolved in toluene (10 mL) under argon condition. Mixture of 2 M K₂CO₃ (3 mL) added to the sealed tube followed by tetrakis(triphenylphosphine)palladium(0) (97 mg, 0.1 eq.). The mixture was purged with argon for 30 min and then heated to 120° C. for 24 h. The progress of the reaction was monitored by TLC (n-hexane-CH₂Cl₂). The crude mixture was extracted with CH₂Cl₂ and combined organic layer dried over Na₂SO₄, filtered and concentrated on a rotary evaporator, the resulting residue was purified by silica column chromatography (100-200 mesh) using n-hexane: CH₂Cl₂ (40:60) as eluent to yield 4 (382 mg, 71%) as a orange coloured liquid. ¹H NMR (400 MHZ, CDCl₃), δ (TMS, ppm): 8.52 (br. d, J=9.1 Hz, 2H), 8.33 (dd, J=8.5, 1.1 Hz, 1H), 7.57-7.64 (m, 2H), 7.32-7.38 (m, 2H), 6.78-6.84 (m, 2H), 5.07-5.15 (m, 1H), 3.00 (s, 6H), 2.14-2.23 (m, 2H), 1.71-1.79 (m, 2H), 1.12-1.28 (m, 36H), 0.76-0.80 ppm (t, 6H).

### Example 6

### Synthesis of (E)-6-(4-(bis(2-ethylhexyl)amino) styryl)-2-(tricosan-12-yl)-1H-benzo[de]isoquinoline-1,3 (2H)-dione (5)

Compound 6-bromo-2-(tricosan-12-yl)-1H-benzo[de]isoquinoline-1,3 (2H)-dione (500 mg, 1.0 eq), Pd(OAc)₂ (18.75 mg, 0.1 eq.), tris-(ortho-tolyl)phosphine (25.42 mg, 0.1 eq.), triethylamine (0.5 ml) and N,N-bis(2-ethylhexyl)-4-vinylaniline (316 mg, 1.1 eq.) in dry DMF (15 ml) was stirred at 120° C. during 16 h under argon atmosphere. The mixture was cooled to ambient temperature then diluted with water and extracted with CH₂Cl₂. The organic layer was dried with Na₂SO₄ and evaporated in vacuum. The resulting residue was purified by silica column chromatography (100-200 mesh) using pet ether: CH₂Cl₂ (40:60) as eluent to yield 5 (531 mg, 74%) as a red coloured liquid. ¹H NMR (500 MHZ, CDCl₃), δ (TMS, ppm): 8.50-8.66 (m, 3H), 7.97 (d, J=8.0 Hz, 1H), 7.75 (t, J=7.9 Hz, 1H), 7.68 (d, J=16.0 Hz, 1H), 7.49-7.56 (d, J=8.9 Hz, 2H), 7.31 (d, J=15.8 Hz, 1H), 6.68-6.75 (d, J=8.9 Hz, 2H), 5.15-5.21 (m, 1H), 3.30 (quind, J=14.8, 7.3 Hz, 4H), 2.17-2.31 (m, 2H), 1.77-1.89 (m, 4H), 1.25-1.43 (m, 28H), 1.20 (br. s., 26H), 0.89-0.95 (m, 12H), 0.86 ppm (t, J=7.0 Hz, 6H).

### Example 7

### 6-((1H-benzo[d][1,2,3]triazol-1-yl)oxy)-2-(tricosan-12-yl)-1H-benzo[de]isoquinoline-1,3 (2H)-dione (6)

A sealed tube was charged with 6-bromo-2-(tricosan-12-yl)-1H-benzo[de]isoquinoline-1,3 (2H)-dione (50 mg, 1 eq.) and 1-hydroxybenzotriazole (17 mg, 1.5 eq.). Both the compounds dissolved in DMF (3 mL) under argon condition. The mixture was purged with argon for 30 min and then heated to 150° C. for 24 h. The progress of the reaction was monitored by TLC (n-hexane-CH₂Cl₂). The crude mixture was extracted with CH₂Cl₂ and combined organic layer dried over Na₂SO₄, filtered and concentrated on a rotary evaporator, the resulting residue was purified by silica column chromatography (100-200 mesh) using n-hexane: CH₂Cl₂ (30:70) as eluent to yield 6 (32 mg, 49%) as a green coloured liquid. ¹H NMR (400 MHZ, CDCl₃), δ (TMS, ppm)=8.98-8.97 (d, 1H), 8.81-8.79 (d, 1H), 8.69-8.65 (m, 2H), 8.27-8.24 (d, 1H), 8.12-8.09 (d, 1H), 7.87-7.83 (t, 1H), 7.74-7.72 (t, 1H), 7.61-7.57 (t, 1H) 5.02-4.92 (m, 1H), 2.29-2.21 (m, 2H), 1.90-1.83 (m, 2H), 1.32-1.20 (m, 36H), 0.84 (t, 6H).

### Example 8

### 6-(benzo[c][1,2,5]thiadiazol-4-ylamino)-2-(tricosan-12-yl)-1H-benzo[de]isoquinoline-1,3 (2H)-dione (7)

Compound 6-bromo-2-(tricosan-12-yl)-1H-benzo[de]isoquinoline-1,3 (2H)-dione (50 mg, 1.0 eq), Pd₂ (dba)₃ (1.8 mg, 0.1 eq.), Tri-tert-butylphosphonium tetrafluoroborate (2.5 mg, 0.1 eq.), sodium t-butoxide 80 mg (10 eq.) and benzo[c][1,2,5]thiadiazol-4-amine (25 mg, 2 eq.) in Toluene (5 ml) was stirred at 120° C. during 16 h under argon atmosphere. The mixture was cooled to ambient temperature then diluted with water and extracted with CH₂Cl₂. The organic layer was dried with Na₂SO₄ and evaporated in vacuum. The resulting residue was purified by silica column chromatography (100-200 mesh) using pet ether: CH₂Cl₂ (30:70) as eluent to yield 7 (36 mg, 64%) as a orange coloured liquid. ¹H NMR (400 MHZ, CDCl₃), δ (TMS, ppm)=8.53-8.73 (m, 2H), 8.47 (d, J=7.9 Hz, 1H), 8.00 (s, 1H), 7.88 (d, J=8.1 Hz, 1H), 7.80 (dd, J=8.4, 7.4 Hz, 1H), 7.55-7.66 (m, 2H), 7.43 (d, J=6.9 Hz, 1H), 5.15-5.23 (m, 1H), 2.20-2.31 (m, 2H), 1.79-1.91 (m, 2H), 1.26-1.21 (m, 36H), 0.86-0.88 ppm (t, 6H).

### Example 9

### 6-(10,11-dihydro-5H-dibenzo[b,f]azepin-5-yl)-2-(tricosan-12-yl)-1H-benzo[de]isoquinoline-1,3 (2H)-dione (8)

Compound 6-bromo-2-(tricosan-12-yl)-1H-benzo[de]isoquinoline-1,3 (2H)-dione (50 mg, 1.0 eq), 10,11-dihydro-5H-dibenzo[b,f]azepine (25 mg, 1.5 eq.), Pd(dba)₂ (4.8 mg, 0.1 eq.), tri(o-tolyl)phosphine (2.5 mg, 0.1 eq.) and potassium t-butoxide 20 mg (2 eq.) in Toluene (5 ml) was stirred at 120° C. during 16 h under argon atmosphere. The mixture was cooled to ambient temperature then diluted with water and extracted with CH₂Cl₂. The organic layer was dried with Na₂SO₄ and evaporated in vacuum. The resulting residue was purified by silica column chromatography (100-200 mesh) using pet ether: CH₂Cl₂ (50:50) as eluent to yield 8 (39 mg, 65%) as a green coloured liquid. ¹H NMR (400 MHZ, CDCl₃), δ (TMS, ppm)=8.40 (dd, J=19.8, 6.9 Hz, 1H), 8.26 (dd, J=19.8, 8.4 Hz, 1H), 7.83-7.90 (m, 1H), 7.53-7.63 (m, 2H), 7.19 (d, 2H), 7.18 (td, J=7.1, 1.9 Hz, 2H), 7.06 (d, J=7.6 Hz, 2H), 6.76 (t, J=8.0 Hz, 2H), 5.07-5.17 (m, 1H), 2.88 (t, 6H), 2.19 (d, J=9.2 Hz, 2H), 1.72-1.84 (m, 2H), 1.26-1.19 (m, 36H), 0.87 ppm (t, 6H).

### Example 10

### 6-(3-chloro-10,11-dihydro-5H-dibenzo[b,f]azepin-5-yl)-2-(tricosan-12-yl)-1H-benzo[de]isoquinoline-1,3 (2H)-dione (9)

Compound 6-bromo-2-(tricosan-12-yl)-1H-benzo[de]isoquinoline-1,3 (2H)-dione (50 mg, 1.0 eq), 3-chloro-10,11-dihydro-5H-dibenzo[b,f]azepine (28 mg, 1.5 eq.), Pd(dba)₂ (4.8 mg, 0.1 eq.), tri(o-tolyl)phosphine (2.5 mg, 0.1 eq.) and potassium t-butoxide 20 mg (2 eq.) in Toluene (5 ml) was stirred at 120° C. during 16 h under argon atmosphere. The mixture was cooled to ambient temperature then diluted with water and extracted with CH₂Cl₂. The organic layer was dried with Na₂SO₄ and evaporated in vacuum. The resulting residue was purified by silica column chromatography (100-200 mesh) using pet ether: CH₂Cl₂ (50:50) as eluent to yield 9 (47 mg, 75%) as a green coloured liquid. ¹H NMR (400 MHZ, CDCl₃), δ (TMS, ppm)=8.40 (dd, J=19.8, 6.9 Hz, 1H), 8.26 (dd, J=19.8, 8.4 Hz, 1H), 7.83-7.90 (m, 1H), 7.53-7.63 (m, 2H), 7.43 (s, 1H), 7.19 (d, 1H), 7.18 (td, J=7.1, 1.9 Hz, 2H), 7.10 (m, 1H), 7.06 (d, J=7.6 Hz, 2H), 5.15-5.23 (m, 1H), 2.91 (t, 6H), 2.20-2.31 (m, 2H), 1.79-1.91 (m, 2H), 1.26-1.21 (m, 36H), 0.86-0.88 ppm (t, 6H).

### Example 11

### 6-(10-methoxy-5H-dibenzo[b,f]azepin-5-yl)-2-(tricosan-12-yl)-1H-benzo[de]isoquinoline-1,3 (2H)-dione (10)

Compound 6-bromo-2-(tricosan-12-yl)-1H-benzo de]isoquinoline-1,3 (2H)-dione (50 mg, 1.0 eq), 10-methoxy-5H-dibenzo[b,f]azepine (28 mg, 1.5 eq.), Pd(dba)₂ (4.8 mg, 0.1 eq.), tri(o-tolyl)phosphine (2.5 mg, 0.1 eq.) and potassium t-butoxide 20 mg (2 eq.) in Toluene (5 ml) was stirred at 120° C. during 16 h under argon atmosphere. The mixture was cooled to ambient temperature then diluted with water and extracted with CH₂Cl₂. The organic layer was dried with Na₂SO₄ and evaporated in vacuum. The resulting residue was purified by silica column chromatography (100-200 mesh) using pet ether: CH₂Cl₂ (50:50) as eluent to yield 10 (43 mg, 69%) as a green coloured liquid. ¹H NMR (400 MHZ, CDCl₃), δ (TMS, ppm)=8.40 (dd, J=19.8, 6.9 Hz, 1H), 8.26 (dd, J=19.8, 8.4 Hz, 1H), 7.83-7.90 (m, 1H), 7.53-7.63 (m, 2H), 7.43-7.50 (m, 3H), 7.38 (td, J=7.1, 1.9 Hz, 2H), 7.31 (d, J=7.6 Hz, 1H), 7.12 (t, J=8.0 Hz, 1H), 6.89 (d, J=9.2 Hz, 1H), 6.20 (s, 1H), 5.07-5.17 (m, 1H), 3.82 (s, 3H), 2.19 (d, J=9.2 Hz, 2H), 1.72-1.84 (m, 2H), 1.26-1.19 (m, 36H), 0.87 ppm (t, 6H).

### Example 12

### 6-(diphenylamino)-2-(tricosan-12-yl)-1H-benzo[de]isoquinoline-1,3 (2H)-dione (11)

Compound 6-bromo-2-(tricosan-12-yl)-1H-benzo de]isoquinoline-1,3 (2H)-dione (50 mg, 1.0 eq), diphenylamine (22 mg, 1.5 eq.), Pd(OAc)₂ (1.9 mg, 0.1 eq.), Tri-tert-butylphosphonium tetrafluoroborate (2.4 mg, 0.1 eq.) and sodium t-butoxide 16 mg (2 eq.) in Toluene (5 ml) was stirred at 120° C. during 16 h under argon atmosphere. The mixture was cooled to ambient temperature then diluted with water and extracted with CH₂Cl₂. The organic layer was dried with Na₂SO₄ and evaporated in vacuum. The resulting residue was purified by silica column chromatography (100-200 mesh) using pet ether: CH₂Cl₂ (50:50) as eluent to yield 11 (38 mg, 66%) as a orange coloured liquid. ¹H NMR (400 MHZ, CDCl₃), δ (TMS, ppm)=8.51 (br. m, 2H), 8.17 (d, J=8.0 Hz, 1H), 7.51 (t, J=7.6 Hz, 1H), 7.39 (d, J=8.0 Hz, 1H), 7.30 (m, 2H), 7.04-7.14 (m, 4H), 6.95 (t, J=7.4 Hz, 4H), 5.18 (br. s., 1H), 2.21-2.30 (m, 2H), 1.80-1.89 (m, 2H), 1.23 (m, 36H), 0.89 ppm (t, 6H).

### Example 13

### 6-(10H-phenothiazin-10-yl)-2-(tricosan-12-yl)-1H-benzo[de]isoquinoline-1,3 (2H)-dione (12)

Compound 6-bromo-2-(tricosan-12-yl)-1H-benzo[de]isoquinoline-1,3 (2H)-dione (50 mg, 1.0 eq), 10H-phenothiazine (25 mg, 1.5 eq.), Pd₂ (dba)₃ (7.6 mg, 0.1 eq.), BINAP (5.2 mg, 0.1 eq.) and Cs₂CO₃₅₅ mg (2 eq.) in Toluene (5 ml) was stirred at 120° C. during 16 h under argon atmosphere. The mixture was cooled to ambient temperature then diluted with water and extracted with CH₂Cl₂. The organic layer was dried with Na₂SO₄ and evaporated in vacuum. The resulting residue was purified by silica column chromatography (100-200 mesh) using pet ether: CH₂Cl₂ (30:70) as eluent to yield 12 (31 mg, 52%) as a greenish yellow coloured liquid. 1H NMR (400 MHZ, CDCl₃), δ (TMS, ppm)=8.79 (d, J=6.2 Hz, 2H), 8.62 (dd, J=0.56 Hz, 5.8 Hz, 1H), 8.49-8.48 (m, 1H), 7.93 (d, J=6.1 Hz, 1H), 7.72 (d, J=6.0 Hz, 1H), 7.08 (dd, J=1.1 Hz, 6.0 Hz, 1H), 6.84 (td, J=0.72 Hz, 6.0 Hz, 2H), 6.75 (td, J=1.2 Hz, 6.7 Hz, 2H), 6.03 (dd, J=0.56 Hz, 6.6 Hz, 2H), 5.18 (br. s., 1H), 2.21-2.30 (m, 2H), 1.80-1.89 (m, 2H), 1.23 (m, 36H), 0.89 ppm (t, 6H).

### Example 14

### 6,7-bis(4-(dimethylamino)phenyl)-2-(2-ethylhexyl)-1H-benzo[de]isoquinoline-1,3 (2H)-dione (13)

A sealed tube was charged with 6,7-dibromo-2-(2-ethylhexyl)-1H-benzo[de]isoquinoline-1,3 (2H)-dione (50 mg, 1 eq.) and (4-(dimethylamino)phenyl) boronic acid (44 mg, 2.5 eq.). Both the compounds dissolved in toluene (10 mL) under argon condition. Mixture of 2 M K₂CO₃ (1 mL) added to the sealed tube followed by tetrak is (triphenylphosphine)palladium(0) (13 mg, 0.1 eq.). The mixture was purged with argon for 30 min and then heated to 120° C. for 24 h. The progress of the reaction was monitored by TLC (n-hexane-CH₂Cl₂). The crude mixture was extracted with CH₂Cl₂ and combined organic layer dried over Na₂SO₄, filtered and concentrated on a rotary evaporator, the resulting residue was purified by silica column chromatography (100-200 mesh) using n-hexane: CH₂Cl₂ (40:60) as eluent to yield 13 (40 mg, 68%) as a red coloured solid. ¹H NMR (400 MHZ, CDCl₃), δ (TMS, ppm)=8.62-8.73 (m, J=7.7 Hz, 2H), 7.66-7.75 (m, J=7.7 Hz, 2H), 6.88 (d, J=8.8 Hz, 4H), 6.35 (d, J=8.8 Hz, 4H), 4.17 (dd, J=7.3, 3.0 Hz, 2H), 2.87 (s, 12H), 1.98-2.05 (m, 1H), 1.31-1.40 (m, 8H), 0.90 (t, 7H).

TABLE 1
Emission lifetime of 1-5 in MTHF solution (C =
10⁻⁵M) and neat state at 298K. Fluorescence lifetime
measurements of 1-5 in solution and neat state showed
different exponential decays with major lifetimes of 1-5 in ns.

| | Solution state | Neat state |
|---|---|---|
| | Fluorescence | Fluorescence |
| Molecules | Lifetime (ns) | Lifetime (ns) |
| 1 | τ₁ = 3.73 (84%) | τ₁ = 12.9 (51%) |
| | τ₂ = 1.92 (11%) | τ₂ = 1.88 (22%) |
| | τ₃ = 0.30 (5%) | τ₃ = 0.55 (27%) |
| 2 | τ₁ = 6.62 (98%) | τ₁ = 24.7 (82%) |
| | τ₂ = 0.23 (2%) | τ₂ = 2.13 (10%) |
| | | τ₃ = 0.82 (8%) |
| 3 | τ₁ = 4.61 (90%) | τ₁ = 5.56 (73%) |
| | τ₂ = 1.44 (06%) | τ₂ = 2.02 (22%) |
| | τ₃ = 0.03 (03%) | τ₃ = 0.31 (5%) |
| 4 | τ₁ = 5.01 (95%) | τ₁ = 7.69 (52%) |
| | τ₂ = 0.64 (3%) | τ₂ = 2.77 (36%) |
| | τ₃ = 0.04 (2%) | τ₃ = 0.52 (12%) |
| 5 | τ₁ = 0.31 (5%) | τ₁ = 1.78 (47%) |
| | τ₂ = 3.36 (95%) | τ₂ = 0.28 (8%) |
| | | τ₃ = 4.50 (45%) |

TABLE 2
Emission lifetime of 1-5 in MTHF solution (C = 1 ×
10⁻⁵M) and TADF lifetime decay in the neat state (before and after
purging with oxygen) at 298K. TADF properties of 1-5 were further
confirmed by the changes in the emission spectra and fluorescence
and TADF lifetime measurements in the presence of oxygen. Here we
noticed that the liquid matrix acts as a supportive layer to protect
from oxygen, and hence the intensity changes and lifetimes are minimum.

| | | Solution state | Neat state TADF |
|---|---|---|---|
| Molecules | Condition | Fluorescence Lifetime (ns) | Lifetime (μs) |
| 1 | Before | τ₁ = 0.54 (20%) | τ₁ = 7.90 (25%) |
| | | τ₂ = 1.85 (71%) | τ₂ = 0.27 (75%) |
| | | τ₃ = 4.69 (9%) | |
| | After | τ₁ = 0.32 (10%) | τ₁ = 0.36 (100%) |
| | | τ₂ = 1.08 (43%) | |
| | | τ₃ = 2.27 (47%) | |
| 2 | Before | τ₁ = 0.84 (23%) | τ₁ = 9.47 (77%) |
| | | τ₂ = 7.27 (77%) | τ₂ = 0.33 (23%) |
| | After | τ₁ = 0.67 (16%) | τ₁ = 8.22 (43%) |
| | | τ₂ = 5.22 (84%) | τ₂ = 0.31 (57%) |
| 3 | Before | τ₁ = 0.41 (4%) | τ₁ = 13.4 (46%) |
| | | τ₂ = 3.72 (17%) | τ₂ = 0.54 (54%) |
| | | τ₃ = 8.34 (79%) | |
| | After | τ₁ = 0.31 (5%) | τ₁ = 11.7 (40%) |
| | | τ₂ = 2.66 (62%) | τ₂ = 0.48 (60%) |
| | | τ₃ = 4.48 (33%) | |
| 4 | Before | τ₁ = 1.32 (3%) | τ₁ = 10.0 (34%) |
| | | τ₂ = 6.42 (95%) | τ₂ = 0.44 (66%) |
| | | τ₃ = 0.16 (2%) | |
| | After | τ₁ = 0.31 (7%) | τ₁ = 8.09 (33%) |
| | | τ₂ = 3.38 (93%) | τ₂ = 0.43 (67%) |
| 5 | Before | τ₁ = 0.34 (5%) | τ₁ = 12.4 (31%) |
| | | τ₂ = 3.59 (95%) | τ₂ = 0.55 (69%) |
| | After | τ₁ = 0.34 (6%) | τ₁ = 11.6 (29%) |
| | | τ₂ = 3.03 (94%) | τ₂ = 0.55 (71%) |

TABLE 3
Emission quantum yield of 1-5 in neat state at 298K and temperature-
dependent TADF lifetime of 1-5 in the neat state. Molecules 1-5
have an excellent quantum yield in neat state as well as in PLA
hybrid sheets it showing the excellent dispersibility of TADF
SOLs in the PLA matrix. Also, molecule 1-5 shows an increase in
TADF lifetime upon increasing the temperature from 77K to 298K.

| | Quantum Yield (%) | | | |
|---|---|---|---|---|
| | Neat | PLA Hybrid | TADF lifetime | |
| Compounds | Liquid | Sheets | Condition | Lifetime (μs) |
| 1 | 66 | 60 | 298K | τ₁ = 7.90 (25%) |
| | | | | τ₂ = 0.27 (75%) |
| | | | 77K | τ₁ = 0.45 (75%) |
| | | | | τ₂ = 0.08 (25%) |
| 2 | 68 | 64 | 298K | τ₁ = 10.1 (45%) |
| | | | | τ₂ = 0.09 (55%) |
| | | | 77K | τ₁ = 0.39 (48%) |
| | | | | τ₂ = 0.05 (52%) |
| 3 | 79 | 76 | 298K | τ₁ = 28.0 (49%) |
| | | | | τ₂ = 0.73 (15%) |
| | | | | τ₃ = 0.04 (36%) |
| | | | 77K | τ₁ = 11.3 (23%) |
| | | | | τ₂ = 0.37 (12%) |
| | | | | τ₃ = 0.04 (65%) |
| 4 | 83 | 79 | 298K | τ₁ = 44.8 (60%) |
| | | | | τ₂ = 5.33 (29%) |
| | | | | τ₃ = 0.10 (11%) |
| | | | 77K | τ₁ = 11.5 (25%) |
| | | | | τ₂ = 0.47 (75%) |
| 5 | 81 | 76 | 298K | τ₁ = 12.1 (31%) |
| | | | | τ₂ = 0.55 (69%) |
| | | | 77K | τ₁ = 2.88 (39%) |
| | | | | τ₂ = 0.19 (61%) |

TABLE 4
Variation of the emission lifetime and TADF lifetime of 1 with increasing
equivalents of 3 in the neat state monitored at 450 nm and 520 nm. Fluorescence
and TADF lifetime of donor 1 gradually reduced upon increasing the concentration
of dopants 3 due to efficient ET from donor to dopants.

| | Fluorescence | Fluorescence | | |
|---|---|---|---|---|
| | Lifetime (ns) | Lifetime (ns) | TADF Lifetime | TADF Lifetime |
| Ratio of | monitored at | monitored at | (μs) monitored | (μs) monitored |
| 1 and 3 | 450 nm | 520 nm | at 450 nm | at 520 nm |
| 1 | τ₁ = 12.9 (51%) | τ₁ = 12.9 (51%) | τ₁ = 7.90 (25%) | τ₁ = 5.96 (29%) |
| | τ₂ = 1.88 (22%) | τ₂ = 1.88 (22%) | τ₂ = 0.27 (75%) | τ₂ = 0.39 (71%) |
| | τ₃ = 0.55 (27%) | τ₃ = 0.55 (27%) | | |
| 1:0.0006 | τ₁ = 1.67 (66%) | τ₁ = 2.18 (62%) | τ₁ = 6.79 (36%) | τ₁ = 7.67 (36%) |
| | τ₂ = 0.44 (20%) | τ₂ = 0.77 (21%) | τ₂ = 0.31 (64%) | τ₂ = 0.40 (64%) |
| | τ₃ = 11.1 (14%) | τ₃ = 14.1 (17%) | | |
| 1:0.0012 | τ₁ = 1.56 (63%) | τ₁ = 3.97 (36%) | τ₁ = 5.02 (18%) | τ₁ = 8.59 (33%) |
| | τ₂ = 0.43 (25%) | τ₂ = 14.4 (29%) | τ₂ = 0.32 (82%) | τ₂ = 0.41 (67%) |
| | τ₃ = 10.6 (12%) | τ₃ = 1.10 (35%) | | |
| 1:0.0024 | τ₁ = 1.04 (54%) | τ₁ = 5.63 (44%) | τ₁ = 4.94 (17%) | τ₁ = 12.4 (48%) |
| | τ₂ = 8.43 (11%) | τ₂ = 15.0 (31%) | τ₂ = 0.35 (83%) | τ₂ = 0.48 (52%) |
| | τ₃ = 0.02 (35%) | τ₃ = 1.26 (25%) | | |
| 1:0.0036 | τ₁ = 0.64 (37%) | τ₁ = 5.37 (40%) | τ₁ = 4.19 (14%) | τ₁ = 0.52 (52%) |
| | τ₂ = 6.97 (11%) | τ₂ = 15.2 (31%) | τ₂ = 0.33 (86%) | τ₂ = 16.2 (48%) |
| | τ₃ = 0.14 (53%) | τ₃ = 1.30 (29%) | | |

TABLE 5
Variation of the emission lifetime and TADF lifetime of 1 with increasing
equivalents of 4 in the neat state monitored at 450 nm and 550 nm. Fluorescence
and TADF lifetime of donor 1 gradually reduced upon increasing the concentration
of dopants 4 due to efficient ET from donor to dopants.

| | Fluorescence | Fluorescence | | |
|---|---|---|---|---|
| | Lifetime (ns) | Lifetime (ns) | TADF Lifetime | TADF Lifetime |
| Ratio of | monitored at | monitored at | (μs) monitored | (μs) monitored |
| 1 and 4 | 450 nm | 550 nm | at 450 nm | at 550 nm |
| 1 | τ₁ = 12.9 (51%) | τ₁ = 12.9 (51%) | τ₁ = 7.90 (25%) | τ₁ = 11.5 (36%) |
| | τ₂ = 1.88 (22%) | τ₂ = 1.88 (22%) | τ₂ = 0.27 (75%) | τ₂ = 0.49 (64%) |
| | τ₃ = 0.55 (27%) | τ₃ = 0.55 (27%) | | |
| 1:0.0006 | τ₁ = 0.34 (7%) | τ₁ = 1.51 (17%) | τ₁ = 7.54 (31%) | τ₁ = 13.7 (38%) |
| | τ₂ = 1.25 (74%) | τ₂ = 4.56 (68%) | τ₂ = 0.35 (69%) | τ₂ = 0.58 (62%) |
| | τ₃ = 5.33 (19%) | τ₃ = 14.0 (15%) | | |
| 1:0.0012 | τ₁ = 0.32 (6%) | τ₁ = 4.44 (67%) | τ₁ = 6.22 (28%) | τ₁ = 0.67 (51%) |
| | τ₂ = 1.13 (71%) | τ₂ = 1.19 (18%) | τ₂ = 0.31 (72%) | τ₂ = 27.6 (49%) |
| | τ₃ = 4.84 (23%) | τ₃ = 15.0 (15%) | | |
| 1:0.0024 | τ₁ = 0.24 (7%) | τ₁ = 4.57 (65%) | τ₁ = 5.83 (29%) | τ₁ = 0.70 (49%) |
| | τ₂ = 0.93 (69%) | τ₂ = 1.32 (20%) | τ₂ = 0.32 (71%) | τ₂ = 28.8 (51%) |
| | τ₃ = 3.97 (24%) | τ₃ = 15.6 (15%) | | |
| 1:0.0036 | τ₁ = 0.53 (55%) | τ₁ = 4.46 (57%) | τ₁ = 5.02 (18%) | τ₁ = 0.72 (51%) |
| | τ₂ = 2.22 (38%) | τ₂ = 16.1 (16%) | τ₂ = 0.32 (82%) | τ₂ = 32.6 (49%) |
| | τ₃ = 0.10 (7%) | τ₃ = 1.21 (27%) | | |

TABLE 6
Variation of the emission lifetime and TADF lifetime of 1
with increasing equivalents of 5 in the neat state monitored
at 450 nm and 580 nm. Fluorescence and TADF lifetime of donor
1 gradually reduced upon increasing the concentration of
dopants 5 due to efficient ET from donor to dopants.

| | Fluorescence | | |
|---|---|---|---|
| | Lifetime | TADF Lifetime | TADF Lifetime |
| Ratio of | (ns) monitored | (μs) monitored | (μs) monitored |
| 1 and 5 | at 450 nm | at 450 nm | at 580 nm |
| 1 | τ₁ = 12.9 (51%) | τ₁ = 7.90 (25%) | τ₁ = 0.61 (58%) |
| | τ₂ = 1.88 (22%) | τ₂ = 0.27 (75%) | τ₂ = 24.7 (42%) |
| | τ₃ = 0.55 (27%) | | |
| 1:0.0008 | τ₁ = 0.32 (7%) | τ₁ = 7.45 (35%) | τ₁ = 0.76 (52%) |
| | τ₂ = 1.22 (75%) | τ₂ = 0.33 (65%) | τ₂ = 33.6 (48%) |
| | τ₃ = 5.18 (18%) | | |
| 1:0.0016 | τ₁ = 0.28 (7%) | τ₁ = 5.58 (26%) | τ₁ = 0.74 (53%) |
| | τ₂ = 1.01 (68%) | τ₂ = 0.33 (74%) | τ₂ = 33.7 (47%) |
| | τ₃ = 4.25 (25%) | | |
| 1:0.0032 | τ₁ = 0.17 (7%) | τ₁ = 5.29 (32%) | τ₁ = 0.73 (53%) |
| | τ₂ = 0.72 (64%) | τ₂ = 0.29 (68%) | τ₂ = 35.3 (47%) |
| | τ₃ = 2.92 (29%) | | |
| 1:0.0048 | τ₁ = 0.51 (56%) | τ₁ = 3.33 (22%) | τ₁ = 0.83 (51%) |
| | τ₂ = 2.20 (39%) | τ₂ = 0.30 (78%) | τ₂ = 37.9 (49%) |
| | τ₃ = 0.14 (5%) | | |

Solvent-free organic liquid (SOL) having attractive TADF features are provided

The SOLs are easy to synthesize

The SOLs can have tunable emission (blue to red)

SOLS are paintable to form large area thin film

SOLs retain high quantum yield upon dispersing in polymers (even at low concentrations

3D printing applications possible.

1-12. (canceled)

13. A solvent-free organic liquid compound according to formula (I): 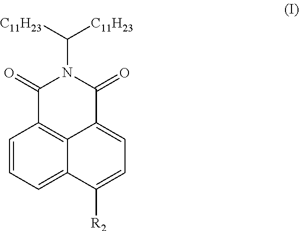
where:
R₂ is independently selected from the group consisting of 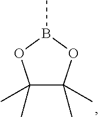
—O—R₃, —NR₄R₅, -Ph-NR₈R₉, 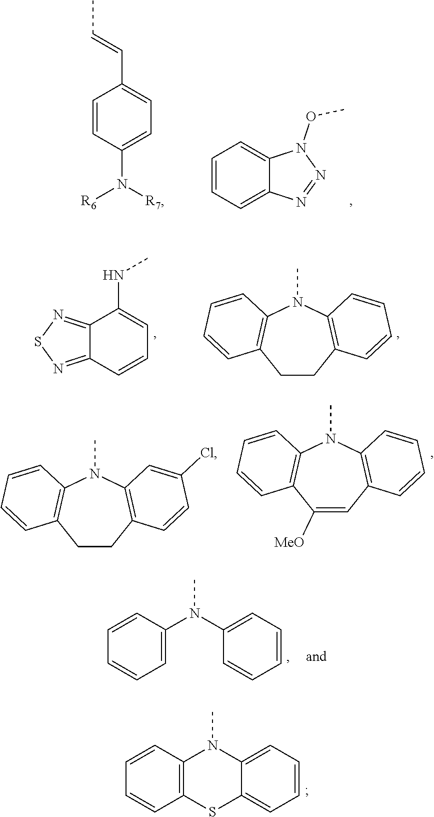
and
R₃, R₄, R₅, R₆, R₇, R₈, and R₉ are independently selected from group consisting of (un)substituted linear or branched alkyl, (un)substituted linear or branched alkylene, (un)substituted linear or branched alkynyl, (un)substituted aryl, and or (un)substituted heteroaryl.

14. The solvent-free organic liquid compound of claim 13, selected from the group consisting of:

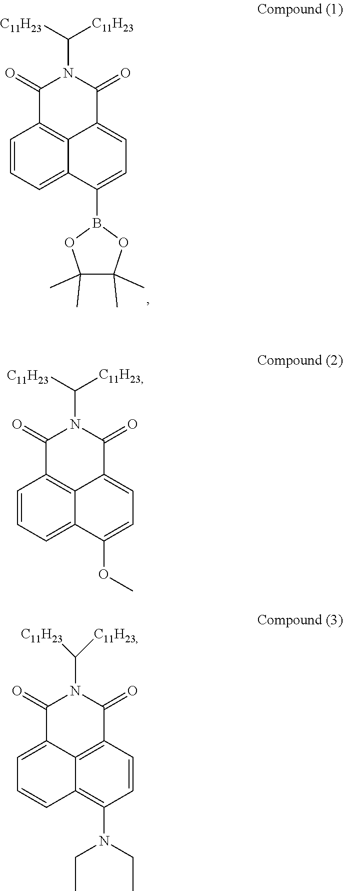

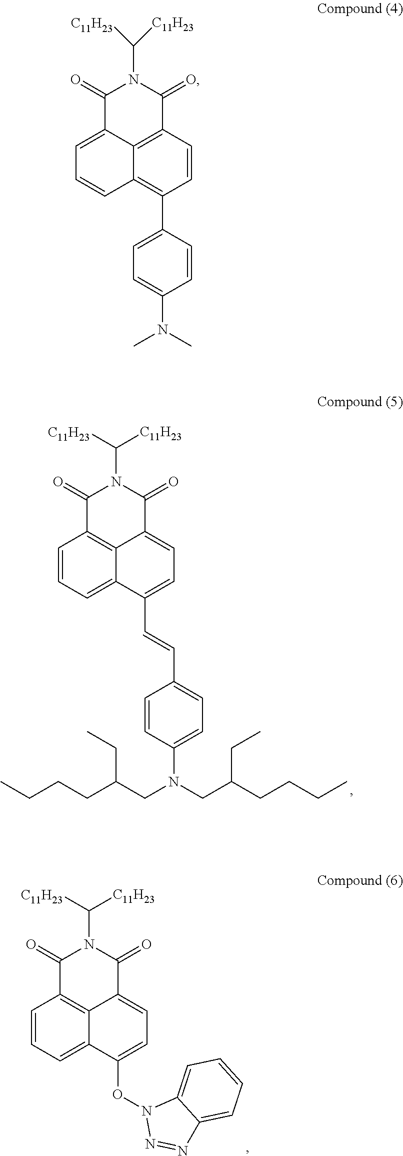

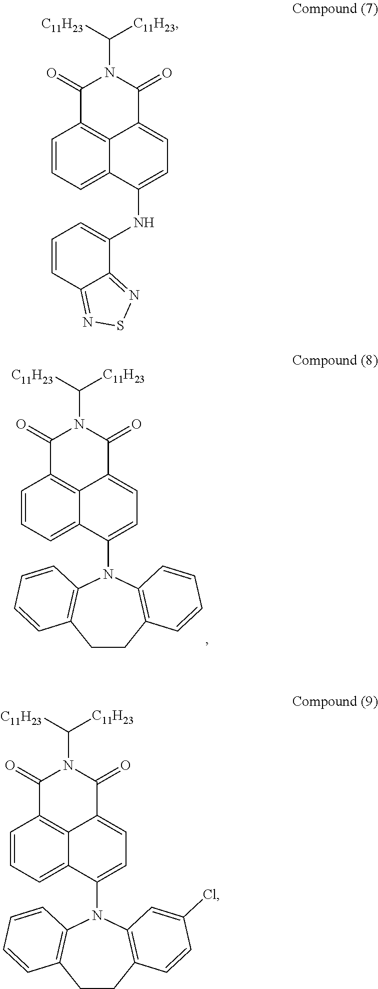

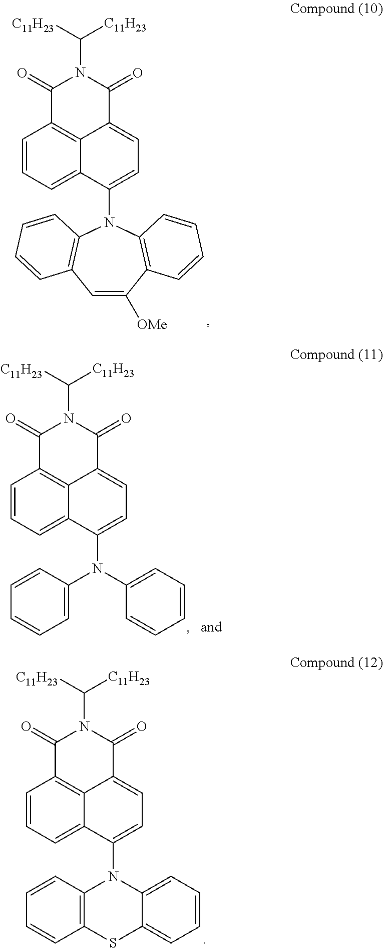

15. A method for preparing the solvent-free organic liquid compound according to claim 13, the method comprising:
reacting compound (Ia): 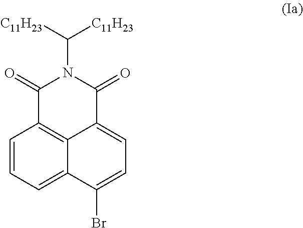
with a reactant in a reaction mixture at 100° C. to 150° C. for 9 hours to 28 hours, to obtain the compound of formula (I),
wherein
the reaction mixture comprises a solvent and a base;
the reactant is selected from the group consisting of 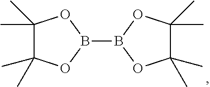
MeOH, 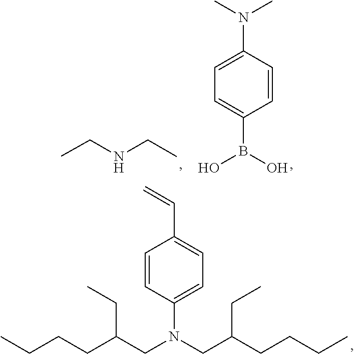
1-hydroxybenzotriazole, tri-tert-butylphosphonium tetrafluoroborate, 10,11-dihydro-5H-dibenzo[b,f]azepine, 3-chloro-10,11-dihydro-5H-dibenzo[b,f]azepine, 10-methoxy-5H-dibenzo[b,f]azepine, diphenylamine, and 10H-phenothiazine.

16. The method according to claim 15, wherein:
the base is selected from the group consisting of KOAc, NaOAc, triethylamine, sodium-t-butoxide, Cs₂CO₃, K₂CO₃, NaOC(CH₃)₃ and KOC(CH₃)₃; and
the solvent is selected from the group consisting of 1,4-dioxane, MeOH, toluene, and DMF.

17. The method according to claim 15, wherein the reaction mixture further comprises a catalyst.

18. The method according to claim 17, wherein:
the catalyst is selected from the group consisting of Pd(dppf)Cl₂, Pd(PPh₃)₄, Pd(OAc)₂, Pd₂(dba)₃, and Pd(dba)₂;
the base is selected from the group consisting of KOAc, NaOAc, triethylamine, sodium-t-butoxide, Cs₂CO₃, K₂CO₃, NaOC(CH₃)₃ and KOC(CH₃)₃; and
the solvent is selected from the group consisting of 1,4-dioxane, MeOH, toluene, and DMF.

19. The method according to claim 18, wherein:
the reaction mixture further comprises a further ingredient selected from the group consisting of tris(o-tolyl)phosphine, tri-tert-butylphosphonium tetrafluoroborate, N,N-bis(2-ethylhexyl)-4-vinylaniline, benzo[c][1,2,5]thiadiazol-4-amine, BINAP, and CuSO₄·5H₂O.

20. The method according to claim 15, wherein:
the reactant is 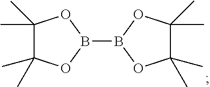
the reaction mixture further comprises a catalyst;
the compound (Ia) is reacted in the reaction mixture at reflux for 14 hours to 18 hours to obtain compound (1): 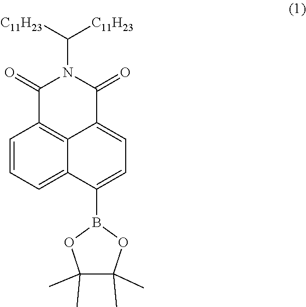
as the compound of formula (I).

21. The method according to claim 15, wherein:
the reactant is MeOH;
the compound (Ia) is reacted with the reactant in the reactant mixture at reflux for 10 hours to 14 hours to obtain compound (2): 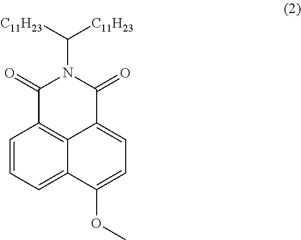
as the compound of formula (I).

22. The method according to claim 15, wherein:
the reactant is 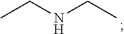
the reaction mixture further comprises CuSO₄·5H₂O;
the solvent is selected from the group consisting of 1,4-dioxane, toluene, and DMF; and
the compound (Ia) is reacted with the reactant in the reaction mixture to obtain compound (3): 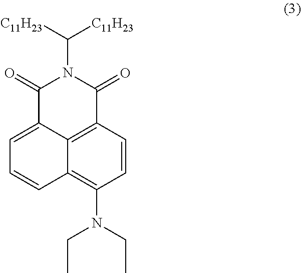
as the compound of formula (I).

23. The method according to claim 15, wherein:
the reactant is 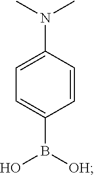
the reaction mixture further comprises a catalyst;
the compound (Ia) is reacted with the reactant at 110° C. to 130° C. for 20 hours to 28 hours to obtain compound (4): 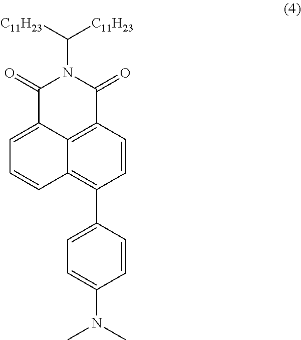
as the compound of formula (I).

24. The method according to claim 23, wherein:
the catalyst is selected from the group consisting of Pd(dppf)Cl₂, Pd(PPh₃)₄, Pd(OAc)₂, Pd₂(dba)₃ and Pd(dba)₂;
the base is selected from the group consisting of KOAc, NaOAc, K₂CO₃, NaOC(CH₃)₃ and KOC(CH₃)₃; and
the solvent is selected from the group consisting of 1,4-dioxane, toluene, and DMF.

25. The method according to claim 15, wherein:
the reactant is 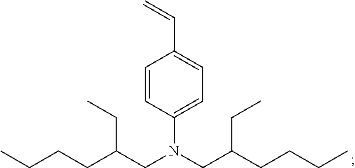 [SMILES: C=Cc1ccc(N(CC(CC)CCCC)CC(CC)CCCC)cc1]
the reaction mixture further comprises a catalyst
the compound (Ia) is reacted with the reactant in the reaction mixture at 110° C. to 130° C. to obtain compound (5): 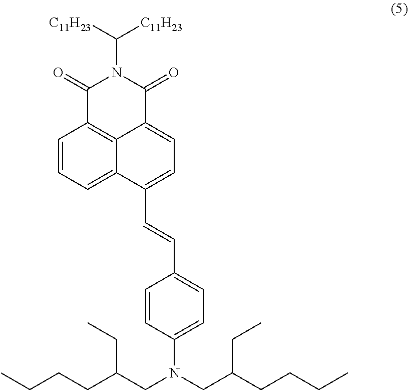
as the compound of formula (I).

26. The method according to claim 25, wherein:
the catalyst is selected from the group consisting of Pd(dppf)Cl₂, Pd(PPh₃)₄, Pd(OAc)₂, Pd₂(dba)₃ and Pd(dba)₂;
the base is selected from the group consisting of KOAc, NaOAc, K₂CO₃, NaOC(CH₃)₃ and KOC(CH₃)₃; and
the solvent is selected from the group consisting of 1,4-dioxane, toluene, and DMF.

27. A composition comprising:
the solvent-free organic liquid compound according to claim 13; and
at least one formulating agent or excipient,
wherein the composition exhibits thermally activated delayed fluorescence with luminescence quantum yields from 60% to 80% and lifetimes of 8 μs to 45 μs.
